(19) United States
(12) Patent Application Publication
(10) Pub. No.: US 2024/0002509 A1
(43) Pub. Date: Jan. 4, 2024

(54) ANTIBODY Fc VARIANTS

(71) Applicant: Novartis AG, Basel (CH)

(72) Inventors: Brian Walter GRANDA, Salisbury, MA (US); Darko SKEGRO, Riehen (CH)

(21) Appl. No.: 18/251,913

(22) PCT Filed: Nov. 4, 2021
(86) PCT No.: PCT/IB2021/060228

§ 371 Date: May 5, 2023

Related U.S. Application Data
(60) Provisional application No. 63/110,490, filed on Nov. 6, 2020.

(51) Int. Cl. C07K 16/28; C12N 15/63

(52) U.S. Cl. CPC C07K 16/2809; C12N 15/63; C07K 2317/24; C07K 2317/71; C07K 2317/31; C07K 2317/732; C07K 2317/734; C07K 2317/524

(57) ABSTRACT

The present invention relates to antibodies comprising Fc variants and their uses. The Fc variants exhibit reduced or undetectable binding to Fc receptors, and reduced or undetectable effector functions. These variants are beneficial for a patient suffering from a disease which could be treated with an antibody for which it is desirable to reduce the effector functions induced by antibodies.

### SEQUENCE LISTING

The instant application contains a Sequence Listing which has been submitted electronically in ASCII format and is hereby incorporated by reference in its entirety. Said ASCII copy, created on Oct. 26, 2021, is named PAT058983-WO-PCT SL.txt and is 42,827 bytes in size.

### FIELD

The present invention is directed to polypeptides comprising variant of Fc region, and compositions and methods of use thereof.

### BACKGROUND

Monoclonal antibodies are very effective biotherapeutics. An important aspect of antibodies is their ability to bind antigens. Therapeutic antibodies may upon binding sequester targets, for instance prevent ligand - receptor interactions and downstream signaling, without the need for any effector functions. Other therapeutic antibodies bind antigen and at the same time recruit immune effector cells via the Fc. To date, all approved recombinant monoclonal antibodies are of the human IgG subclass, which can engage both humoral and cellular components of the immune system. The cellular immune response occurs mostly due to the interactions between the antibody and Fc gamma receptors (FcγR). Intracellular signaling through the activating receptors, FcγR1A, 2A, and 3A is modulated through the phosphorylation of immuno-receptor tyrosine-based activation motifs (ITAMs), which leads to effector functions such as antibody-dependent cell-mediated cytotoxicity (ADCC), antibody-dependent cellular phagocytosis (ADCP), and inflammation via the induction of cytokine secretion. Antibody mediated complement activation is regulated via Fc interaction with the complement component C1q and can trigger complement dependant cytotoxicity (CDC).

The Fc region of an antibody is limited in variability and is involved in effecting the physiological roles played by the antibody. The effector functions attributable to the Fc region of an antibody vary with the class and subclass of antibody and include binding of the antibody via the Fc region to a specific Fc receptor on a cell which triggers various biological responses. These receptors are expressed in a variety of immune cells including monocytes, macrophages, neutrophils, dendritic cells, eosinophils, mast cells, platelets, B cells, large granular lymphocytes, Langerhans' cells, natural killer (NK) cells, and T cells. Formation of the Fc/FcγR complex recruits these effector cells to sites of bound antigen, typically resulting in signaling events within the cells and important subsequent immune responses such as release of inflammation mediators, B cell activation, endocytosis, phagocytosis, and cytotoxic attack. In addition, an overlapping site on the Fc region of the molecule also controls the activation of a cell independent cytotoxic function mediated by complement, otherwise known as complement dependent cytotoxicity (CDC).

In many circumstances, the binding and stimulation of effector functions mediated by the Fc region of immunoglobulins is highly beneficial or even essential, for instance for binding to tumor antigens on the cell surface of malignant transformed cells, e.g. for a CD20 antibody. However, in other instances it may be more advantageous to decrease or even to fully eliminate the effector functions. This is particularly true for those antibodies designed to deliver a drug (e.g., toxins and isotopes) to the target cell where the Fc/FcγR mediated effector functions bring healthy immune cells into the proximity of the deadly payload, resulting in depletion of normal lymphoid tissue along with the target cells (Hutchins, et al., PNAS USA 92 (1995) 11980-11984; White, et al., Annu Rev Med 52 (2001) 125-145). In these cases, the use of antibodies that poorly recruit complement or effector cells would be of a tremendous benefit (see also, Wu, et al., Cell Immunol 200 (2000) 16-26; Shields, et al., J. Biol Chem 276(9) (2001) 6591-6604; U.S. Pat. Nos. 6,194,551; 5,885,573 and PCT publication WO 04/029207).

For cases where mAbs are intended to engage cell surface receptors and prevent receptor-ligand interactions (for example antagonists, e.g. antagonists of cytokines), it may be desirable to reduce or eliminate effector function for example to prevent target cell death or unwanted cytokine secretion. Other examples where reduced effector function may be warranted include preventing antibody-drug conjugates from interacting with FcγRs leading to off-target cytotoxicity. The need for reducing or eliminating effector function was recognized with the first approved mAb, the anti-CD3ε mAb, OKT3, muromonab, which was intended to prevent T cell activation in tissue transplant patients receiving a donor kidney, lung, or heart (Chatenoud and Bluestone, 2007). Many patients receiving muromonab had adverse events including the induction of pro-inflammatory cytokines (for example cytokine storm), which was attributed in part to muromonab's interactions with FcγR's (Alegre et al., 1992). In order to reduce this unintended effector function, a human IgG1 variant L234A/L235A has been generated (Xu et al., 2000), which showed reduced inflammatory cytokine release. Reduced affinity of antibodies to the FcγRII receptor in particular would be advantageous for antibodies inducing platelet activation and aggregation via FcγRII receptor binding, which would be a serious side-effect of such antibodies.

Although there are certain subclasses of human immunoglobulins that lack specific effector functions, there are no known naturally occurring immunoglobulins that lack all effector functions.

Silenced effector functions can be obtained by mutation in the Fc region of the antibodies and have been described in the art: LALA and N297A (Strohl, W., 2009, Curr. Opin. Biotechnol. vol. 20(6):685-691); and D265A (Baudino et al., 2008, J. Immunol. 181: 6664-69) see also Heusser et al., WO2012065950. Among the four IgG subclasses, each has a different ability to elicit immune effector functions. For instance, IgG1 and IgG3 recruit complement more effectively than IgG2 and IgG4 (Tao et al., 1993). Additionally, IgG2 and IgG4 have very limited ability to elicit ADCC (Brerski et al., 2014). Therefore, several investigators have employed a cross-subclass approach to reduce effector functions. In a further refinement of the cross-subclass approach, An et al. generated an IgG2 variant with point mutations from IgG4 (i.e., H268Q/V309L/A330S/P331S). This variant had reduced effector function (An et al., 2009). In a similar approach, a variant was reported that contained the IgG2 to IgG4 cross-subclass mutations V309L/A330S/P331S combined with the non-germline mutations V234A/G237A/P238S/H268A, which resulted in undetectable CDC, ADCC, and ADCP (Vafa et al., 2014). Other investigators replaced amino acids in the Fc region with a different amino acid residues such that the antibody has altered C1 q binding and/or reduced or abolished complement dependent cytotoxicity (CDC). This approach is described in, e.g., U.S. Pat. No. 6,194,551 by Idusogie et al. Examples of silent Fc lgG1 antibodies include the LALA mutant comprising L234A and L235A mutation in the lgG1 Fc amino acid sequence. Another example of a silent lgG₁ antibody is the DAPA (D265A, P329A) mutation (U.S. Pat. No. 6,737,056). Another silent lgG1 antibody comprises the N297A mutation, which results in aglycosylated/non-glycosylated antibodies. Other alternate approaches to engineer or mutate critical residues in the Fc region that are responsible for effector functions have been reported. For examples see PCT publications WO 2009/100309 (Medimmune), WO 2006/076594 (Xencor), US 2006/0134709 (Macrogenics), U.S. Pat. No. 6,737,056 (Genentech), US 2010/0166740 (Roche) and WO2019068632 (Janssen).

### SUMMARY

There is an unmet need for therapeutic antibodies with a strongly decreased or diminished effector functions whilst at the same time have excellent pharmacokinetic properties such as stability in formulations, with prolonged shelf life, as well as a longer in vivo and in vitro half-life, robust recombinant expression levels and suitability for large scale manufacturing and purification. In addition, there is a need for highly silenced Fc containing proteins and antibodies that retain the N297 glycosylation site. Antibody glycosylation, in particular fucosylation, presence of terminal galactose, high mannose or sialylation, are all involved in modulating effector functions such as ADCC activation pathways, but also in protein stability and half-life (reviewed in Boune S et al., Antibodies, 9, 22, 2020). The IgG1 N297 position is often mutated, for instance to Alanine, or Glutamine, to abolish N-glycosylation and thereby down modulate effector functions (Bolt S, Routledge E, Lloyd I, et al (1993) Eur J Immunol. 1993; 23:403-411).

Retaining N297 and thereby retaining Fc N-glycosylation can be beneficial for stabilizing Fc containing protein molecules; in particular stabilizing correct pairing of heavy chains in antibodies, increasing solubility, stability, and decreasing protein aggregation propensity, facilitating increased shelf life, in-vitro and in-vivo half-life of silenced Fc containing therapeutic protein molecules. Half-life of a number of glycoproteins can be enhanced by sialylation; sialic acid acts as a cap that hides the penultimate galactose residue recognized by the hepatic asialoglycoprotein receptor (ASGPR). Therefore, in order for a silenced Fc containing molecule to have an extended half-life via high sialylation, which can be mediated via specific high sialytion providing expression systems and cell lines, it is necessary to maintain the N-glycosylation attachment site at Arginine 297 in the Fc part of the molecule.

The present invention provides binding molecules, for example, antibodies, comprising IgG1 Fc variants with unexpected strongly reduced and/or undetectable binding to all Fc gamma receptors and strongly reduced and/or undetectable binding to C1q, resulting in a strongly reduced or even undetectable effector functions, including ADCC, CDC, and ADCP whilst preferably maintaining capability for normal N-glycosylation

Accordingly, the present invention is directed to binding molecules comprising a human IgG1 Fc variant of a wild-type human IgG1 Fc region and one or more antigen binding domains, wherein the Fc variant comprises amino acid substitutions selected from the combinations of substitutions: L234A, L235A, G237A (LALAGA), substitutions L234A, L235A, S267K, P329A (LALASKPA), subsitutions D265A, P329A, S267K (DAPASK), substitutions G237A, D265A, P329A (GADAPA), substitutions G237A, D265A, P329A, S267K (GADAPASK), substitutions L234A, L235A, P329G (LALAPG), substitutions L234A, L235A, P329A (LALAPA), wherein the amino acid residues are numbered according to the EU index of Kabat.

In some aspect of the invention, the Fc variants comprises the nucleic acid sequences of SEQ ID NO's 8, 10, 12, 16, 18, 20 or 22, listed in Table 1 below, or any sequence having at least about 90%, 91% 92%, 93%, 94%, 95%, 96%, 97%, 98%, 99%, or 100% homology thereto.

A particularly preferred embodiment of the invention the binding molecule comprises a human IgG1 Fc variant of a wild-type human IgG1 Fc region and one or more antigen binding domains, wherein the Fc variant comprises the preferred amino acid substitutions L234A, L235A, S267K, P329A (LALASKPA) in SEQ ID NO 15, or substitutions G237A, D265A, P329A, S267K (GADAPASK) in SEQ ID NO 21, wherein the amino acid residues are numbered according to the EU index of Kabat.

In some aspect of the invention, the Fc variant comprises the sequence of SEQ ID NO: 21 (see Table 1 below), or a sequence having at least about 90%, 91% 92%, 93%, 94%, 95%, 96%, 97%, 98%, 99%, or 100% homology thereto.

In some aspect of the invention, the Fc variant comprises the sequence of SEQ ID NO: 15 (see Table 1 below), or a sequence having at least about 90%, 91% 92%, 93%, 94%, 95%, 96%, 97%, 98%, 99%, or 100% homology thereto.

In some aspect of the invention, the binding molecule is a human or humanized IgG1 monoclonal antibody.

In some aspect of the invention, the binding molecule has reduced or undetectable binding affinity to a Fc gamma receptor or C1q compared to a polypeptide comprising the wild-type human IgG1 Fc region optionally measured by surface plasmon resonance using a Biacore T200 instrument, wherein the Fc gamma receptor is selected from the group consisting of Fc gamma RIA, Fc gamma RIIIa V158 variant and Fc gamma RIIIa F158 variant, and wherein the binding compared to wildtype is reduced by 50%, 80%, 90%, 95%, 98%, 99% or undetectable.

In some aspect of the invention, the modified Fc containing binding molecule binds at least one antigen wherein the antigen is a cell surface antigen.

In some aspect of the invention, the modified Fc containing binding molecule binds at least one antigen that is a secreted or soluble antigen.

In some aspect of the invention, the modified Fc containing binding molecule has reduced or undetectable effector function compared to a polypeptide comprising the wild-type human IgG1 Fc region.

In some aspect of the invention, the modified Fc containing binding molecule is capable of binding to an antigen without triggering detectable antibody-dependent cell-mediated cytotoxicity (ADCC), antibody-dependent cellular phagocytosis (ADCP), or complement dependent cytotoxicity (CDC).

In some aspect of the invention, the modified Fc containing binding molecule is a multi-specific antibody comprising binding domains for two or more antigens.

In some aspect of the invention, the modified Fc containing binding molecule is a bi-specific antibody comprising binding domains for two antigens.

In some aspect of the invention, the modified Fc containing binding molecule further comprises knob in hole mutations.

Another aspect of the invention are methods of treating a disease in an individual, wherein it the effector function of the binding molecule is reduced or undetectable in the individual compared to the effector function induced by a polypeptide comprising the wild-type human IgG1 Fc region, the method comprising administering the binding molecule disclosed herein to the individual. In another aspect the invention provides modified Fc containing binding molecules for use in human therapy.

In some aspect of the invention, the effector function to be reduced or diminished is antibody-dependent cell-mediated cytotoxicity (ADCC) in the individual. In some aspect of the invention, the effector function to be reduced or diminished is antibody-dependent cellular phagocytosis (ADCP) in the individual. In some aspect of the invention, the effector function to be reduced or diminished is complement dependent cytotoxicity (CDC) in the individual.

Additionally disclosed herein are compositions comprising Fc modified binding molecules according to the invention. In some aspect of the invention, the composition further comprising a pharmaceutically acceptable carrier.

Additionally disclosed herein are isolated polynucleotide encoding the binding molecule comprising a modified IgG1 Fc sequence according to the invention. In some aspect of the invention, the isolated polynucleotide comprises the modified IgG1 Fc sequence of SEQ ID NO:16, or a sequence having at least about 90%, 91% 92%, 93%, 94%, 95%, 96%, 97%, 98%, 99%, or 100% homology thereto. In other aspect of the invention, the isolated polynucleotide comprises the modified IgG1 Fc sequence of SEQ ID NO:22, or a sequence having at least about 90%, 91% 92%, 93%, 94%, 95%, 96%, 97%, 98%, 99%, or 100% homology thereto.

Additionally disclosed herein are vectors comprising the polynucleotides encoding modified Fc containing binding molecules of the invention.

Additionally disclosed herein are host cells comprising vectors or polynucleotides encoding and capable of expressing modified Fc containing binding molecules of the invention.

### BRIEF DESCRIPTION OF THE DRAWINGS

FIG. 1A and B shows schematic overview of a biacore measuring cycle.

FIG. 2 shows representative sensorgrams and response and concentration plots. FIG. 2A shows representative sensorgrams and response plots of WT, LALAPA-IgG1, LALAGA-IgG1, LALAPG-IgG1, DAPA-IgG1, LALASKPA-IgG1, DAPASK-IgG1, GADAPA-IgG1, GADAPASK-IgG1 and DANAPA-IgG1. FIG. 2 B shows sensorgrams and binding kinetics of WT, LALAPA-IgG1, LALAGA-IgG1, LALAPG-IgG1, DAPA-IgG1, LALASKPA-IgG1, DAPASK-IgG1, GADAPA-IgG1, GADAPASK-IgG1 and DANAPA-IgG1 towards FcγR3A V158 and FIG. 2 C shows sensorgrams and binding kinetics of WT, LALAPA-IgG1, LALAGA-IgG1, LALAPG-IgG1, DAPA-IgG1, LALASKPA-IgG1, DAPASK-IgG1, GADAPA-IgG1, GADAPASK-IgG1 and DANAPA-IgG1 towards C1q.

FIG. 3A shows the nuclear factor of activated T-cells (NFAT) pathway activity of the wild type and mutated antibodies. FIG. 3 B shows the NFAT pathway activity of the wild type and mutated antibodies, cells sensitized with addition of INFgamma.

### DETAILED DESCRIPTION

### General Matters

In order that the present invention may be more readily understood, certain terms are defined throughout the detailed description. Unless defined otherwise, all technical and scientific terms used herein have the same meaning as commonly understood by those of ordinary skill in the art to which this invention pertains.

Unless stated otherwise, the following terms and phrases as used herein are intended to have the following meanings:

The term “binding molecule” as used herein refers to a molecule that binds to a target molecule, for example, an antigen, and has reduced or undetectable binding affinity to an Fc receptor or C1q. A “reduced binding affinity to a Fc gamma receptor or C1q” as used herein refers to a reduction of binding affinity to an Fc gamma receptor or C1q in comparison to a control (for example a polypeptide with a wildtype Fc region), by at least 20%; a “strongly reduced binding affinity to a Fc gamma receptor or C1q” as used herein refers to a reduction of binding affinity to a Fc gamma receptor, in comparison to a control, by at least 50%; and an “undetectable binding affinity to a Fc gamma receptor or C1q” as used herein refers to binding affinity to a Fc gamma receptor or C1q that is below the detection limit of the assay being used. In some embodiments, the binding affinity is measured by surface plasmon resonance using a Biacore T200 instrument.

The binding molecule of the present disclosure encompass antibodies, antibody variants, fragments of antibodies, antigen binding portions of antibodies that can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). The binding molecule also encompasses nanobodies, Fabs, DARPins, avimers, affibodies, and anticalins.

The term “antibody” as used herein refers to a polypeptide of the immunoglobulin family that is capable of binding a corresponding antigen non-covalently, reversibly, and in a specific manner. For example, a naturally occurring IgG antibody is a tetramer comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and components of the classical complement system.

The term “antibody” includes, but is not limited to, monoclonal antibodies, human antibodies, humanized antibodies, camelid antibodies, chimeric antibodies, and anti-idiotypic (anti-Id) antibodies (including, e.g., anti-Id antibodies to antibodies of the present disclosure) and functional fragments or fusions thereof. The antibodies can be of any isotype/class (e.g., IgG, IgE, IgM, IgD, IgA and IgY), or subclass (e.g., IgG₁, IgG₂, IgG₃, IgG₄, IgA₁ and IgA₂) . Antibodies according to the invention comprise at least one modified and silenced IgG1 Fc fragment.

“Complementarity-determining domains” or “complementarity-determining regions” (“CDRs”) interchangeably refer to the hypervariable regions of V_{L} and V_{H}. The CDRs are the target protein-binding site of the antibody chains that harbors specificity for such target protein. There are three CDRs (CDR1-3, numbered sequentially from the N-terminus) in each human V_{L} or V_{H}, constituting in total about 15-20% of the variable domains. CDRs can be referred to by their region and order. For example, “VHCDR1” or “HCDR1” both refer to the first CDR of the heavy chain variable region. The CDRs are structurally complementary to the epitope of the target protein and are thus directly responsible for the binding specificity. The remaining stretches of the V_{L} or V_{H}, the so-called framework regions, exhibit less variation in amino acid sequence (Kuby, Immunology, 4th ed., Chapter 4. W.H. Freeman & Co., New York, 2000).

The positions of the CDRs and framework regions can be determined using various well-known definitions in the art, e.g., Kabat, Chothia, IMGT, AbM, and combined definitions (see, e.g., Johnson et al., Nucleic Acids Res., 29:205-206 (2001); Chothia and Lesk, J. Mol. Biol., 196:901-917 (1987); Chothia et al., Nature, 342:877-883 (1989); Chothia et al., J. Mol. Biol., 227:799-817 (1992); Lefranc, M. P., Nucleic Acids Res., 29:207-209 (2001); Al-Lazikani et al., J. Mol. Biol., 273:927-748 (1997)). Definitions of antigen combining sites are also described in the following: Ruiz et al., Nucleic Acids Res., 28:219-221 (2000); MacCallum et al., J. Mol. Biol., 262:732-745 (1996); and Martin et al., Proc. Natl. Acad. Sci. USA, 86:9268-9272 (1989); Martin et al., Methods Enzymol., 203:121-153 (1991); and Rees et al., In Sternberg M. J. E. (ed.), Protein Structure Prediction, Oxford University Press, Oxford, 141-172 (1996). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a V_{H}, e.g., a mammalian V_{H}, e.g., a human V_{H}; and amino acid residues 24-34 (LCDR1), 50-56 (LCDR2), and 89-97 (LCDR3) in a V_{L}, e.g., a mammalian V_{L}, e.g., a human V_{L}. Under IMGT the CDR amino acid residues in the V_{H} are numbered approximately 26-35 (CDR1), 51-57 (CDR2) and 93-102 (CDR3), and the CDR amino acid residues in the V_{L} are numbered approximately 27-32 (CDR1), 50-52 (CDR2), and 89-97 (CDR3) (numbering according to “Kabat”). Under IMGT, the CDR regions of an antibody can be determined using the program IMGT/DomainGap Align. IMGT tools are available at world wide web (www).imgt.org.

Both the light and heavy chains are divided into regions of structural and functional homology. The terms “constant” and “variable” are used functionally. In this regard, it will be appreciated that the variable domains of both the light (V_{L}) and heavy (V_{H}) chain portions determine antigen recognition and specificity. Conversely, the constant domains of the light chain (CL) and the heavy chain (CH1, CH2, or CH3) confer important biological properties such as secretion, transplacental mobility, Fc receptor binding, complement binding, and the like. By convention, the numbering of the constant region domains increases as they become more distal from the antigen-binding site or amino-terminus of the antibody. The N-terminus is a variable region and at the C-terminus is a constant region; the CH3 and CL domains actually comprise the carboxy-terminal domains of the heavy and light chain, respectively.

The terms “antigen-binding domain” and “antigen-binding fragment” are used interchangeably, and refer to one or more portions of an antibody that retain the ability to specifically interact with (e.g., by binding, steric hindrance, stabilizing/destabilizing, spatial distribution) an epitope of an antigen. Examples of binding fragments include, but are not limited to, single-chain Fvs (scFv), disulfide-linked Fvs (sdFv), F(ab)₂ fragment, Fab fragment, F(ab′)₂, fragment F(ab′) fragments, a monovalent fragment consisting of the V_{L}, V_{H}, CL and CH1 domains; a bivalent fragment comprising two Fab fragments linked by a disulfide bridge at the hinge region; an Fd fragment consisting of the V_{H} and CH1 domains (and optionally a portion of the hinge); an Fv fragment consisting of the V_{L} and V_{H} domains of a single arm of an antibody; a dAb fragment (Ward et al., Nature 341:544-546, 1989), which consists of a V_{H} domain; and an isolated complementarity determining region (CDR), or other epitope-binding fragments of an antibody.

Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (“scFv”); see, e.g., Bird et al., Science 242:423-426, 1988; and Huston et al., Proc. Natl. Acad. Sci. 85:5879-5883, 1988). Such single chain antibodies are also intended to be encompassed within the term “antigen-binding fragment.” Often there is a peptide linker between the V_{H} and V_{L} domains. In preferred embodiments, scFvs of the disclosure have the general structures: NH₂-V_{L}-linker-V_{H}-COOH or NH₂-V_{H}-linker-V_{L}-COOH. These antigen-binding fragments are obtained using conventional techniques known to those of skill in the art, and the fragments are screened for utility in the same manner as are intact antibodies.

Antigen-binding fragments can also be incorporated into single domain antibodies, maxibodies, minibodies, nanobodies, intrabodies, diabodies, triabodies, tetrabodies, v-NAR, and bis-scFv (see, e.g., Hollinger and Hudson, Nature Biotechnology 23:1126-1136, 2005). Antigen-binding fragments can be grafted into scaffolds based on polypeptides such as fibronectin type III (Fn3) (see U.S. Pat. No. 6,703,199, which describes fibronectin polypeptide monobodies).

Antigen-binding fragments can be incorporated into single chain molecules comprising a pair of tandem Fv segments (V_{H}-CH1-V_{H}-CH1) which, together with complementary light chain polypeptides, form a pair of antigen-binding regions (Zapata et al., Protein Eng. 8:1057-1062, 1995; and U.S. Pat. No. 5,641,870).

The term “monoclonal antibody” or “monoclonal antibody composition” as used herein refers to polypeptides, including antibodies and antigen-binding fragments that have substantially identical amino acid sequence or are derived from the same genetic source. This term also includes preparations of antibody molecules of single molecular composition. A monoclonal antibody composition displays a single binding specificity and affinity for a particular epitope. Methods for generation of monoclonal antibodies using phage display technology are known in the art (Proetzel, G., Ebersbach, H. (Eds.) Antibody Methods and Protocols. Humana Press ISBN 978-1-61779-930-3; 2012).

The term “human antibody,” as used herein, includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., J. Mol. Biol. 296:57-86, 2000). In a preferred embodiment, the binding molecule of the present disclosure is a human antibody.

The human antibodies of the present disclosure can include amino acid residues not encoded by human sequences (e.g., mutations introduced by random or site-specific mutagenesis in vitro or by somatic mutation in vivo, or a conservative substitution to promote stability or manufacturing).

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al. 1984, Proc. Natl. Acad. Sci. USA, 81:6851-6855; Morrison and Oi, 1988, Adv. Immunol., 44:65-92; Verhoeyen et al. 1988, Science, 239:1534-1536; Padlan 1991, Molec. Immun., 28:489-498; and Padlan 1994, Molec. Immun., 31:169-217. Other examples of human engineering technology include, but are not limited to Xoma technology disclosed in U.S. Pat. No. 5,766,886. In some embodiments, the binding molecule of the present disclosure is a humanized or chimeric antibody.

Chimeric or humanized antibodies of the present disclosure can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,530,101; 5,585,089; 5,693,762 and 6,180,370 to Queen et al.

The terms “recognize” or “bind” as used herein refers to a binding molecule, an antibody or antigen-binding fragment thereof that finds and interacts (e.g., binds or recognizes) its epitope, whether that epitope is linear, discontinuous or conformational. The term “epitope” refers to a site on an antigen to which an antibody or antigen-binding fragment of the disclosure specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art, for example, x-ray crystallography and 2-dimensional nuclear magnetic resonance (see, e.g., Epitope Mapping Protocols in Methods in Molecular Biology, Vol. 66, G. E. Morris, Ed. (1996)), or electron microscopy. A “paratope” is the part of the antibody which recognizes the epitope of the antigen.

The phrase “specifically binds” or “selectively binds,” when used in the context of describing the interaction between an antigen (e.g., a protein) and an antibody, antibody fragment, or antibody-derived binding agent, refers to a binding reaction that is determinative of the presence of the antigen in a heterogeneous population of proteins and other biologics, e.g., in a biological sample, e.g., a blood, serum, plasma or tissue sample. Thus, under certain designated immunoassay conditions, the antibodies or binding agents with a particular binding specificity bind to a particular antigen at least two times the background and do not substantially bind in a significant amount to other antigens present in the sample. In one aspect, under designated immunoassay conditions, the antibody or binding agent with a particular binding specificity binds to a particular antigen at least ten (10) times the background and does not substantially bind in a significant amount to other antigens present in the sample. Specific binding to an antibody or binding agent under such conditions may require the antibody or agent to have been selected for its specificity for a particular protein. As desired or appropriate, this selection may be achieved by subtracting out antibodies that cross-react with molecules from other species (e.g., mouse or rat) or other subtypes. Alternatively, in some aspects, antibodies or antibody fragments are selected that cross-react with certain desired molecules.

In some embodiments, specific binding of an antibody or antigen-binding fragment of the disclosure means binding with an equilibrium constant (KA) (kₒₙ/k_{off}) of at least 10²M⁻, at least 5×10²M⁻¹, at least 10³M⁻¹, at least 5×10³M⁻¹, at least 10⁴M⁻¹ at least 5×10⁴M⁻¹, at least 10⁵M⁻¹, at least 5×10⁵M⁻¹, at least 10⁶M⁻¹, at least 5×10⁶M⁻¹, at least 10⁷M⁻¹, at least 5×10⁷M⁻¹, at least 10⁸M⁻¹, at least 5×10⁸M⁻¹, at least 10⁹M⁻¹, at least 5×10⁹M⁻¹, at least 10¹⁰M⁻¹, at least 5×10¹⁰M⁻¹, at least 10¹¹M⁻¹, at least 5×10¹¹M⁻¹, at least 10¹²M⁻¹, at least 5×10¹²M⁻¹, at least 10¹³M⁻¹, at least 5×10¹³M⁻¹, at least 10¹⁴M⁻¹, at least 5×10¹⁴M⁻¹, at least 10¹⁵M⁻¹, or at least 5×10¹⁵M⁻¹.

In some embodiments, specific binding of an antibody or antigen-binding fragment of the disclosure means a dissociation rate constant (K_{D}) (k_{off}/kₒₙ) of less than 5×10⁻²M, less than 10⁻² M, less than 5×10⁻³ M, less than 10⁻³ M, less than 5×10 -4 M, less than 10⁻⁴ M, less than 5×10⁻⁵ M, less than 10⁻⁵ M, less than 5×10⁻⁶ M, less than 10⁻⁶ M, less than 5×10⁻⁷ M, less than 10⁻⁷M, less than 5×10⁻⁸ M, less than 10⁻⁸ M, less than 5×10⁻⁹ M, less than 10⁻⁹ M, less than 5×10⁻¹⁰M, less than 10⁻¹⁰M, less than 5×10⁻¹¹ M, less than 10⁻¹¹ M, less than 5×10⁻¹² M, less than 10⁻¹² M, less than 5×10⁻¹³ M, less than 10⁻¹³ M, less than 5×10⁻¹⁴ M, less than 10⁻¹⁴ M, less than 5×10⁻¹⁵ M, or less than 10⁻¹⁵ M or lower, and binds to the target antigen with an affinity that is at least two-fold greater than its affinity for binding to a non-specific antigen (e.g., HSA).

The term “affinity” as used herein refers to the strength of interaction between antibody and antigen at single antigenic sites. Within each antigenic site, the variable region of the antibody “arm” interacts through weak non-covalent forces with antigen at numerous sites; the more interactions, the stronger the affinity. As used herein, the term “high affinity” for an IgG antibody or fragment thereof (e.g., a Fab fragment) refers to an antibody having a K_{D} of 10⁻⁸ M or less, 10⁻⁹ M or less, or 10⁻¹⁰ M, or 10⁻¹¹ M or less, or 10⁻¹² M or less, or 10⁻¹³ M or less for a target antigen. However, high affinity binding can vary for other antibody isotypes. For example, high affinity binding for an IgM isotype refers to an antibody having a K_{D} of 10′ M or less, or 10⁻⁸ M or less. One suitable assay for measuring affinity, e.g., K_{D} involves the use of the BIACORE technology (e.g. by using the BIACORE 3000 instrument (BIACORE, Uppsala, Sweden, or the Biacore T200, GE Healthcare)), which can measure the extent of interactions using surface plasmon resonance technology.

As used herein, the term “avidity” refers to an informative measure of the overall stability or strength of the antibody-antigen complex. It is controlled by three major factors: antibody epitope affinity; the valence of both the antigen and antibody; and the structural arrangement of the interacting parts. Ultimately these factors define the specificity of the antibody, that is, the likelihood that the particular antibody is binding to a precise antigen epitope.

The term “isolated antibody” refers to an antibody that is substantially free of other antibodies having different antigenic specificities. An isolated antibody that specifically binds to one antigen may, however, have cross-reactivity to other antigens. Moreover, an isolated antibody may be substantially free of other cellular material and/or chemicals.

The term “corresponding human germline sequence” refers to the nucleic acid sequence encoding a human variable region amino acid sequence or subsequence that shares the highest determined amino acid sequence identity with a reference variable region amino acid sequence or subsequence in comparison to all other all other known or inferred variable region amino acid sequences encoded by human germline immunoglobulin variable region sequences. The corresponding human germline sequence can also refer to the human variable region amino acid sequence or subsequence with the highest amino acid sequence identity with a reference variable region amino acid sequence or subsequence in comparison to all other evaluated variable region amino acid sequences. The corresponding human germline sequence can be framework regions only, complementarity determining regions only, framework and complementary determining regions, a variable segment (as defined above), or other combinations of sequences or subsequences that comprise a variable region. Sequence identity can be determined using the methods described herein, for example, aligning two sequences using BLAST, ALIGN, or another alignment algorithm known in the art. The corresponding human germline nucleic acid or amino acid sequence can have at least about 90%, 91% 92%, 93%, 94%, 95%, 96%, 97%, 98%, 99%, or 100% sequence identity with the reference variable region nucleic acid or amino acid sequence.

A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically, a specific or selective binding reaction will produce a signal at least twice over the background signal and, more typically, at least 10 to 100 times over the background.

The term “equilibrium dissociation constant (K_{D}, M)” refers to the dissociation rate constant (k_{d}, time⁻¹) divided by the association rate constant (kₐ, time⁻¹, M⁻¹). Equilibrium dissociation constants can be measured using any known method in the art. The antibodies and fragments of the present disclosure generally will have an equilibrium dissociation constant of less than about 10⁻⁷ or 10⁻⁸ M, for example, less than about 10⁻⁹ M or 10⁻¹⁰ M, in some aspects, less than about 10⁻¹¹ M, 10⁻¹² M or 10⁻¹³ M.

The term “bioavailability” refers to the systemic availability (i.e., blood/plasma levels) of a given amount of drug administered to a patient. Bioavailability is an absolute term that indicates measurement of both the time (rate) and total amount (extent) of drug that reaches the general circulation from an administered dosage form.

A “modification” or “mutation” or “substitution” of an amino acid residue/position, as used herein, refers to a change of a primary amino acid sequence as compared to a starting amino acid sequence (e.g., a wild-type sequence), wherein the change results from a sequence alteration involving said amino acid residue/positions. For example, typical modifications include substitution of the residue (or at said position) with another amino acid (e.g., a conservative or non-conservative substitution), insertion of one or more amino acids adjacent to said residue/position, and deletion of said residue/position. An “amino acid mutation,” or variation thereof, refers to the replacement of an existing amino acid residue in a predetermined (starting) amino acid sequence with a different amino acid residue. Generally and preferably, the modification results in alteration in at least one physicobiochemical activity of the variant polypeptide compared to a polypeptide comprising the starting (or “wild type”) amino acid sequence. For example, in the case of an antibody, a physicobiochemical activity that is altered can be binding affinity, binding capability and/or binding effect upon a target molecule.

The term “comprising” encompasses “including” as well as “consisting,” e.g., a composition “comprising” X may consist exclusively of X or may include something additional, e.g., X+Y.

Unless otherwise specifically stated or clear from context, as used herein, the term “about” in relation to a numerical value is understood as being within the normal tolerance in the art, e.g., within two standard deviations of the mean. Thus, “about” can be within +/−10%, 9%, 8%, 7%, 6%, 5%, 4%, 3%, 2%, 1%, 0.1%, 0.05%, or 0.01% of the stated value, preferably +/−10% of the stated value. When used in front of a numerical range or list of numbers, the term “about” applies to each number in the series, e.g., the phrase “about 1-5” should be interpreted as “about 1-about 5”, or, e.g., the phrase “about 1, 2, 3, 4” should be interpreted as “about 1, about 2, about 3, about 4, etc.”

As used herein, “selecting” and “selected” in reference to a patient is used to mean that a particular patient is specifically chosen from a larger group of patients due to the particular patient having a predetermined criterion. Similarly, “selectively treating a patient” refers to providing treatment to a patient that is specifically chosen from a larger group of patients due to the particular patient having a predetermined criteria. Similarly, “selectively administering” refers to administering a drug to a patient that is specifically chosen from a larger group of patients due to the particular patient having a predetermined criterion.

The word “substantially” does not exclude “completely,” e.g., a composition which is “substantially free” from Y may be completely free from Y. Where necessary, the word “substantially” may be omitted from the definition of the disclosure.

As used herein, the phrase “consisting essentially of” refers to the genera or species of active pharmaceutical agents included in a method or composition, as well as any excipients inactive for the intended purpose of the methods or compositions. In some aspects, the phrase “consisting essentially of” expressly excludes the inclusion of one or more additional active agents other than a binding molecule of the present disclosure. In some aspects, the phrase “consisting essentially of” expressly excludes the inclusion of one or more additional active agents other than a binding molecule of the present disclosure and a second co-administered agent.

The term “amino acid” refers to naturally occurring, synthetic, and unnatural amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For example, the codons GCA, GCC, GCG, and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. The following eight groups contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)). In some aspects, the term “conservative sequence modifications” are used to refer to amino acid modifications that do not significantly affect or alter the binding characteristics of the antibody containing the amino acid sequence.

The term “optimized” as used herein refers to a nucleotide sequence that has been altered to encode an amino acid sequence using codons that are preferred in the production cell or organism, generally a eukaryotic cell, for example, a yeast cell, a Pichia cell, a fungal cell, a Trichoderma cell, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence.

The terms “percent identical” or “percent identity,” in the context of two or more nucleic acids or polypeptide sequences, refers to the extent to which two or more sequences or subsequences that are the same. Two sequences are “identical” if they have the same sequence of amino acids or nucleotides over the region being compared. Two sequences are “substantially identical” if two sequences have a specified percentage of amino acid residues or nucleotides that are the same (i.e., 60% identity, optionally 65%, 70%, 75%, 80%, 85%, 90%, 95%, 98% or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 30 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman, Adv. Appl. Math. 2:482c (1970), by the homology alignment algorithm of Needleman and Wunsch, J. Mol. Biol. 48:443 (1970), by the search for similarity method of Pearson and Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Brent et al., Current Protocols in Molecular Biology, 2003).

Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as a basis for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=-4 and a comparison of both strands. For amino acid sequences, the BLASTP program uses as defaults a word length of 3, and expectation (E) of 10, and the BLOSUM62 scoring matrix (see Henikoff and Henikoff, (1989) Proc. Natl. Acad. Sci. USA 89:10915) alignments (B) of 50, expectation (E) of 10, M=5, N=-4, and a comparison of both strands.

The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The percent identity between two amino acid sequences can also be determined using the algorithm of E. Meyers and W. Miller, (Comput. Appl. Biosci. 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch, (J. Mol. Biol. 48:444-453, 1970), algorithm which has been incorporated into the GAP program in the GCG software package (available from University of South Florida), using either a BLOSUM 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. Examples of nucleic acids that are part of the disclosure include cDNA, genomic DNA, recombinant DNA, and RNA (e.g., mRNA). The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., (1991) Nucleic Acid Res. 19:5081; Ohtsuka et al., (1985) J. Biol. Chem. 260:2605-2608; and Rossolini et al., (1994) Mol. Cell. Probes 8:91-98).

The term “operably linked” in the context of nucleic acids refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

As used herein, phrases such as “a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

“IC₅₀” (half-maximal inhibitory concentration) refers to the concentration of a particular antibody or fragment thereof which inhibits a signal halfway (50%) between the baseline control and the maximum possible signal.

“EC₅₀” (half-maximal effective concentration) refers to the concentration of a particular antibody or fragment thereof which induces a response halfway (50%) between the baseline control and the maximum possible effect after a specific exposure or treatment time. For example, the EC5o is the concentration of antibody at which virus infection is reduced by 50%.

“EC₉₀” refers to the concentration of a particular antibody or fragment thereof which induces a response corresponding to 90% of the maximum possible effect after a specific exposure or treatment time. For example, the EC₉₀ is the concentration of an antibody or fragment thereof at which virus infection is reduced by 90%.

The term “treatment” or “treat” is herein defined as the application or administration of an antibody or antigen-binding fragment according to the disclosure comprising an Fc variant, or a pharmaceutical composition comprising said antibody, to a subject or to an isolated tissue or cell line from a subject, where the subject has a particular disease (e.g., arthritis), a symptom associated with the disease, or a predisposition towards development of the disease (if applicable), where the purpose is to cure (if applicable), delay the onset of, reduce the severity of, alleviate, ameliorate one or more symptoms of the disease, improve the disease, reduce or improve any associated symptoms of the disease or the predisposition toward the development of the disease. The term “treatment” or “treat” includes treating a patient suspected to have the disease as well as patients who are ill or who have been diagnosed as suffering from the disease or medical condition, and includes suppression of clinical relapse. The phrase “reducing the likelihood” refers to delaying the onset or development or progression of a disease, infection or disorder.

The term “therapeutically acceptable amount” or “therapeutically effective amount” or “therapeutically effective dose” interchangeably refer to an amount sufficient to effect the desired result (e.g., a reduction in disease activity, inhibition of disease progression, etc.). In some aspects, a therapeutically acceptable amount does not induce or cause undesirable side effects. A therapeutically acceptable amount can be determined by first administering a low dose, and then incrementally increasing that dose until the desired effect is achieved. A “prophylactically effective dosage,” and a “therapeutically effective dosage,” of the molecules of the present disclosure can prevent the onset of, or result in a decrease in severity of, respectively, disease symptoms.

The term “co-administer” refers to the simultaneous presence of two active agents in the blood of an individual. Active agents (e.g., additional therapeutic agents) that are co-administered with the disclosed antibodies and antigen-binding fragments can be concurrently or sequentially delivered.

As used herein, the term “Fc” or “Fc region” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc region” includes both amino acids 231-447 (CH2-CH3) or 216-447 (hinge-CH2-CH3), or fragments thereof. An “Fc fragment” in this context can contain fewer amino acids from either or both of the N- and C-termini but still retains the ability to form a dimer with another Fc region as can be detected using standard methods, generally based on size (e.g., non-denaturing chromatography, size exclusion chromatography). . Unless otherwise specified herein, numbering of amino acid residues in the Fc region or constant region is according to the EU numbering system, also called the EU index, as described in Kabat, et al., Sequences of Proteins of Immunological Interest, 5th Ed. Public Health Service, National Institutes of Health, Bethesda, Md. (1991).

A “variant Fc region” or “modified Fc fragment” comprises an amino acid sequence which differs from that of a “native” or “wildtype” sequence Fc region by virtue of at least one “amino acid modification” as herein defined. Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The term “Fc variant” as used herein refers to a polypeptide comprising a modification in an Fc region. The Fc variants of the present invention are defined according to the amino acid modifications that compose them. Thus, for example, P329G is an Fc variant with the substitution of proline with glycine at position 329 relative to the parent Fc polypeptide, wherein the numbering is according to the EU index. The identity of the wildtype amino acid may be unspecified, in which case the aforementioned variant is referred to as P329G. For all positions discussed in the present invention, numbering is according to the EU index. The EU index or EU index as in Kabat or EU numbering scheme refers to the numbering of the EU antibody (Kabat, et al., Sequences of Proteins of Immunological Interest, 5th Ed. Public Health Service, National Institutes of Health, Bethesda, Md. (1991)).

The modification can be an addition, deletion, or substitution. Substitutions can include naturally occurring amino acids and non-naturally occurring amino acids. Variants may comprise non-natural amino acids. Examples include U.S. Pat. No. 6,586,207; WO 98/48032; WO 03/073238; US 2004/0214988 A1; WO 05/35727 A2; WO 05/74524 A2; Chin, J. W., et al., Journal of the American Chemical Society 124 (2002) 9026-9027; Chin, J. W., and Schultz, P. G., ChemBioChem 11 (2002) 1135-1137; Chin, J. W., et al., PICAS United States of America 99 (2002) 11020-11024; and, Wang, L., and Schultz, P. G., Chem. (2002) 1-10, all entirely incorporated by reference.

The term “Fc region containing polypeptide” refers to a polypeptide, such as a binding molecule, an antibody or immunoadhesin, which comprises an Fc region.

The terms “Fc receptor” or “FcR” are used to describe a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review in Daëron, M., Annu. Rev. Immunol. 15 (1997) 203-234). FcRs are reviewed in Ravetch, and Kinet, Annu. Rev. Immunol 9 (1991) 457-492; Capel, et al., Immunomethods 4 (1994) 25-34; and de Haas, et al., J. Lab. Clin. Med. 126 (1995) 330-41. Other FcRs, including those to be identified in the future, are encompassed by the term “FcR” herein.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRs, FcγRs, FcγRs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis, et al., Immunological Reviews 190 (2002) 123-136, entirely incorporated by reference). Fc binding molecules may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIA, FcγRIB, and FcγRIC; FcγRII (CD32), including isoforms FcγRIIA (including allotypes H131 and R131), FcγRIIB (including FcγRIIB-1 and FcγRIIB-2), and FcγRIIe; and FcγRIII (CD16), including isoforms FcγRIIIA (including allotypes V158 and F158) and FcγRIIIb (including allotypes FcγRIIB-NA1 and FcγRIIB-NA2) (Jefferis, et al., Immunol Lett 82 (2002) 57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “wildtype polypeptide” as used herein is meant an unmodified polypeptide that is subsequently modified to generate a variant. The wildtype polypeptide may be a naturally occurring polypeptide, or a variant or engineered version of a naturally occurring polypeptide. Wildtype polypeptide may refer to the polypeptide itself, compositions that comprise the parent polypeptide, or the amino acid sequence that encodes it. Accordingly, by “wildtype immunoglobulin” as used herein is meant an unmodified immunoglobulin polypeptide that is modified to generate a variant, and by “wildtype antibody” as used herein is meant an unmodified antibody that is modified to generate a variant antibody. It should be noted that “wildtype antibody” includes known commercial, recombinantly produced antibodies as outlined below.

As used herein, the term “antibody effector function(s),” or “effector function” as used herein refers to a function contributed by an Fc effector domain(s) of an IgG (e.g., the Fc region of an immunoglobulin). Such function can be effected by, for example, binding of an Fc effector domain(s) to an Fc receptor on an immune cell with phagocytic or lytic activity or by binding of an Fc effector domain(s) to components of the complement system. Typical effector functions are ADCC, ADCP and CDC. Effector function may also include Fc mediated inflammation and immunomodulation through the induction of cellular differentiation and activation.

As used herein, the term “ADCC” or “antibody dependent cell cytotoxicity” activity refers to a cell-mediated reaction in which nonspecific cytotoxic cells that express FcRs (e.g. Natural Killer (NK) cells, neutrophils, and macrophages) recognize bound antibody on a target cell and subsequently cause lysis of the target cell. The primary cells for mediating ADCC, NK cells, express FcγRIII only, whereas monocytes express FcγRI, FcγRII and FcγRIII FcR expression on hematopoietic cells is summarized in Table 3 on page 464 of Ravetch, and Kinet, Annu. Rev. Immunol 9 (1991) 457-492.

As used herein, the term “ADCP” or “antibody-dependent cellular phagocytosis” refers to a process by which antibody-coated cells are internalized, either in whole or in part, by phagocytic immune cells (e.g., macrophages, neutrophils and dendritic cells) that bind to an immunoglobulin Fc region.

As used herein, the term “CDC” or “complement dependent cytotoxicity” activity refers to a mechanism for inducing cell death in which an Fc effector domain(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

As used herein, “C1q” is a polypeptide that includes a binding site for the Fc region of an immunoglobulin. C1q together with two serine proteases, C1r and C1s, forms the complex C1, the first component of the complement dependent cytotoxicity (CDC) pathway. Human C1q can be purchased commercially from, e.g. Quidel, San Diego, Calif.

A “reduced effector function” as used herein refers to a reduction of a specific effector function, for example ADCC or CDC, in comparison to a control (for example a polypeptide with a wildtype Fc region), by at least 20%, a “strongly reduced effector function” as used herein refers to a reduction of a specific effector function, like for example ADCC or CDC, in comparison to a control, by at least 50%, and an “undetectable effector function” as used herein refers to a redunction of a specific effector function, for example ADCC or CDC that is below the detectable limit of the assay being used.

As used herein, “human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source thereof, e.g. from blood or PBMCs as described herein.

As used herein, the term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived there from without regard to the number of passages. Progeny may not be completely identical in nucleic acid content to a parent cell, but may contain mutations. Mutant progeny that have the same function or biological activity as screened or selected for in the originally transformed cell are included herein.

### Fc Silencing Mutations

The present invention provides binding molecules comprising an modified IgG1 Fc, e.g. antibodies or functional fragments thereof, Fc fusion molecules or multispecific antibody formats, with mutations in the Fc region resulting in “Fc silent” binding molecules that have minimal interaction with effector cells. In general, the “IgG1 Fc region” is used to define the C-terminal region of an immunoglobulin heavy chain, including native sequence Fc region and variant Fc regions. The human IgG1 heavy chain Fc region is generally defined as comprising the C-terminal part of heavy chain starting from amino acid residue in position C226 or from P230 to the carboxyl-terminus of the IgG1 antibody; (K447). The numbering of residues in the Fc region is that of the EU index of Kabat. The C- terminal lysine (residue K447) of the Fc region may be partially or fully removed, due to enzymatic clipping for example, during production or purification of the antibody.

The present invention provides Fc silent antibodies or Fc containing binding proteins or Fc fusion proteins that comprise an IgG1 Fc having a combination of amino acid substitutions selected from the group consisting of substitutions: L234A, L235A, G237A (LALAGA), substitutions L234A, L235A, S267K, P329A (LALASKPA), substitutions D265A, P329A, S267K (DAPASK), substitutions G237A, D265A, P329A (GADAPA), substitutions G237A, D265A, P329A, S267K (GADAPASK), substitutions L234A, L235A, P329G (LALAPG), or substitutions L234A, L235A, P329A (LALAPA), and wherein the amino acid residues are numbered according to the EU index of Kabat. Most preferred embodiments are the IgG1 Fc's containing either LALASKPA and/or GADAPASK silencing motifs.

Fc silent antibodies in the present disclosure result in undetectable or heavily reduced effector functions. For example, an Fc silent antibody according to the present disclosure exhibits ADCC that is below 50% specific cell lysis as compared to the wild type antibody (low ADCC activity), or that is below 30%, 20%, 10%, 5%, 2% or 1% specific cell lysis or that is below the detection limit of the assay being used (un detectable ADCC activity). At the same time, Fc silenced antibodies and binding molecules in the present disclosure retain good developability characteristics; have a high melting temperature of the Fc, are stable, capable of being recombinantly produced at high yields and formulated to high concentrations that remain stable for prolonged periods without aggregation.

### Additional Mutations and Modifications

The binding molecules in the present disclosure may further comprise mutations and/or modifications to improve the properties of the binding molecule.

In one aspect, further modifications are made to decrease the immunogenicity of the binding molecule.

For example, one approach is to “back-mutate” one or more additional framework residues to the corresponding germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back-mutated” to the germline sequence by, for example, site-directed mutagenesis. Such “back-mutated” antibodies are also intended to be encompassed.

Another type of framework modification involves mutating one or more residues within the framework region, or even within one or more CDR regions, to remove T-cell epitopes to thereby reduce the potential immunogenicity of the antibody. This approach is also referred to as “deimmunization” and is described in further detail in U.S. Patent Publication No. 2003/0153043 by Carr et al.

In another aspect, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another aspect, the Fc hinge region of an antibody or fragment is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another aspect, one or more amino acid residues are altered to thereby alter the ability of the antibody to fix complement. This approach is described in, e.g., the PCT Publication WO 94/29351 by Bodmer et al. In a specific aspect, one or more amino acids of an antibody or antigen-binding fragment thereof of the present disclosure are replaced by one or more allotypic amino acid residues, for the IgG₁ subclass and the kappa isotype. Allotypic amino acid residues also include, but are not limited to, the constant region of the heavy chain of the IgG₁, IgG₂, and IgG₃ subclasses as well as the constant region of the light chain of the kappa isotype as described by Jefferis et al., MAbs. 1:332-338 (2009).

In another aspect, the antibody or fragment is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. In a preferred embodiment the modified IgG1 containing binding molecules disclosed herein further comprises modifications to the Fc to include the “YTE” mutations (M252Y, S254T, T256E (according to EU Numbering)) for half-life extension.

### Production of Antibodies and Fragments

The binding molecule of the present invention can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

Also disclosed herein are isolated nucleic acid molecules, or a set of nucleic acid molecules, encoding an antibody or antigen-binding fragment as described herein. In some embodiments, the isolated nucleic acid molecule is complementary DNA (cDNA) or messenger RNA (mRNA).

The disclosure further provides polynucleotides encoding the antibodies and binding molecules described herein, e.g., polynucleotides encoding heavy or light chain variable regions or segments comprising the complementarity determining regions as described herein.

The modified IgG 1 Fc regions of the binding molecules are encoded by the nucleic acid sequences of SEQ ID Nos: 16 and 22 of Table 1. In some aspects, the polynucleotide encoding the Fc regions of the binding molecule has at least 85%, 89%, 90%, 91%, 92%, 93%, 94%, 95%, 96%, 97%, 98%, 99%, or 100% nucleic acid sequence identity with a polynucleotide of SEQ ID NO:16 or 22 (Table 1).

The polynucleotide sequences can be produced by de novo solid-phase DNA synthesis or by PCR mutagenesis of an existing sequence. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H.A. Erlich (Ed.), Freeman Press, NY, NY, 1992; PCR Protocols: A Guide to Methods and Applications, Innis et al. (Ed.), Academic Press, San Diego, CA, 1990; Mattila et al., Nucleic Acids Res. 19:967, 1991; and Eckert et al., PCR Methods and Applications 1:17, 1991.

Also provided in the present disclosure are expression vectors and host cells for producing the binding molecules described above. Disclosed herein are cloning and expression vectors comprising one or more nucleic acid molecules, or set of nucleic acid molecules, encoding the binding molecules described above, wherein the vector is suitable for the recombinant production of the antibody or antigen-binding fragment thereof.

Various expression vectors can be employed to express the polynucleotides encoding the disclosed binding molecules, for example, antibodies. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat Gen. 15:345, 1997). For example, nonviral vectors useful for expression of polynucleotides and polypeptides of the binding molecules in mammalian (e.g., human) cells include pThioHis A, B & C, pcDNA3.1/His, pEBVHis A, B & C (Invitrogen, San Diego, CA), MPSV vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding binding molecules, for example, antibodies. In some aspects, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements may also be required or desired for efficient expression of a binding molecule, for example, an antibody. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted antibody or fragment sequences. More often, the inserted antibody or fragment sequences are linked to a signal sequences before inclusion in the vector. Vectors to be used to receive sequences encoding antibody or fragment V_{H} and V_{L} sometimes also encode constant regions or parts thereof. Such vectors allow expression of the variable regions as fusion proteins with the constant regions thereby leading to production of intact antibodies or fragments thereof. Typically, such constant regions are human.

Disclosed herein are host cells comprising one or more cloning or expression vectors as described herein. The host cells for harboring and expressing the binding molecules can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express the binding molecules. Insect cells in combination with baculovirus vectors can also be used.

In other aspects, mammalian host cells are used to express and produce the binding molecules of the present disclosure. For example, they can be either a hybridoma cell line expressing endogenous immunoglobulin genes (e.g., the myeloma hybridoma clones) or a mammalian cell line harboring an exogenous expression vector. These include any normal mortal or normal or abnormal immortal animal or human cell. For example, a number of suitable host cell lines capable of secreting intact immunoglobulins have been developed, including the CHO cell lines, various COS cell lines, HeLa cells, HEK293 or HEK293T cells, SP2/0 cells, NS0 myeloma cell lines, transformed B-cells and hybridomas. CHO lines are most preferred. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express the binding molecules, can be prepared using expression vectors which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

In some embodiments, the binding molecule is comprised of a single polypeptide chain that is encoded by a single nucleic acid, which may be inserted into a single cloning or expression vectors. In other embodiments, the binding molecule is comprised of two polypeptide chains encoded by more than one nucleic acid, which is referred to herein as “a set of nucleic acid molecules”. In some embodiments, the nucleic acid encoding the first chain is inserted into a first cloning or expression vector and the nucleic acid encoding the second chain is inserted into a second cloning or expression vector. In this situation, the binding molecule is expressed via a set of cloning or expression vectors. Alternatively, both nucleic acids may be inserted into a single cloning or expression vector.

Disclosed herein is a process for the production of the binding molecule as described herein, comprising culturing a host cell as described herein under conditions sufficient to express said antibody or antigen-binding fragment thereof, and thereafter purifying and recovering said antibody or antigen-binding fragment thereof from the host cell culture as one polynucleotide chain.

### Isolation of Recombinant Antibodies and Fragments

A variety of methods of screening antibodies and proteins comprising an antigen-binding portion thereof have been described in the Art. Such methods may be divided into in vivo systems, such as transgenic mice capable of producing fully human antibodies upon antigen immunization and in vitro systems, consisting of generating antibody DNA coding libraries, expressing the DNA library in an appropriate system for antibody production, selecting the clone that express antibody candidate that binds to the target with the affinity selection criteria and recovering the corresponding coding sequence of the selected clone. These in vitro technologies are known as display technologies, and include without limitation, phage display, RNA or DNA display, ribosome display, yeast or mammalian cell display. They have been well described in the Art (for a review see for example: Nelson et al. 2010, Nature Reviews Drug discovery, “Development trends for human monoclonal antibody therapeutics” (Advance Online Publication) and Hoogenboom et al. 2001, Method in Molecular Biology 178:1-37, O'Brien et al., ed., Human Press, Totowa, N.J.). In one specific embodiment, human recombinant antibodies of the disclosure are isolated using phage display methods for screening libraries of human recombinant antibody libraries, such as HuCAL® libraries.

Repertoires of V_{H} and V_{L} genes or related CDR regions can be separately cloned by polymerase chain reaction (PCR) or synthesized by DNA synthesizer and recombined randomly in phage libraries, which can then be screened for antigen-binding clones. Such phage display methods for isolating human antibodies are established in the art or described in the examples below. See for example: U.S. Pat. Nos. 5,223,409; 5,403,484; and 5,571,698 to Ladner et al.; U.S. Pat. Nos. 5,427,908 and 5,580,717 to Dower et al.; U.S. Pat. Nos. 5,969,108 and 6,172,197 to McCafferty et al.; and U.S. Pat. Nos. 6,521,404; 6,544,731; 6,555,313; 6,582,915 and 6,593,081 to Griffiths et al.

In a certain embodiment, human antibodies can be identified using transgenic or transchromosomic mice carrying parts of the human immune system rather than the mouse system. These transgenic and transchromosomic mice include mice referred to herein as HUMAB mice and KM mice, respectively, and are collectively referred to herein as “human Ig mice.”

The HUMAB mouse® (Medarex, Inc.) contains human immunoglobulin gene miniloci that encode un-rearranged human heavy (μ and γ) and κ light chain immunoglobulin sequences, together with targeted mutations that inactivate the endogenous μ and κ chain loci (see e.g., Lonberg, et al. 1994, Nature 368:856-859). Accordingly, the mice exhibit reduced expression of mouse IgM or κ, and in response to immunization, the introduced human heavy and light chain transgenes undergo class switching and somatic mutation to generate high affinity human IgGκ monoclonal (Lonberg, N. et al. 1994, supra; reviewed in Lonberg, N., 1994 Handbook of Experimental Pharmacology 113:49-101; Lonberg, N. and Huszar, D. 1995, Intern. Rev. Immunol. 13:65-93, and Harding, F. and Lonberg, N. 1995, Ann. N. Y. Acad. Sci. 764:536-546). The preparation and use of HUMAB mice, and the genomic modifications carried by such mice, is further described in Taylor, L. et al. 1992, Nucleic Acids Research 20:6287-6295; Chen, J. et at. 1993, International Immunology 5:647-656; Tuaillon et al. 1993, Proc. Natl. Acad. Sci. USA 94:3720-3724; Choi et al. 1993, Nature Genetics 4:117-123; Chen, J. et al. 1993, EMBO J. 12: 821-830; Tuaillon et al. 1994, J. Immunol. 152:2912-2920; Taylor, L. et al. 1994, International Immunology 579-591; and Fishwild, D. et al. 1996, Nature Biotechnology 14: 845-851. See further, U.S. Pat. Nos. 5,569,825; 5,625,126; 5,633,425; 5,789,650; 5,877,397; 5,661,016; 5,814,318; 5,874,299; and 5,770,429; all to Lonberg and Kay; U.S. Pat. No. 5,545,807 to Surani et al.; PCT Publication Nos. WO 92103918, WO 93/12227, WO 94/25585, WO 97113852, WO 98/24884 and WO 99/45962, all to Lonberg and Kay; and PCT Publication No. WO 01/14424 to Korman et al.

In another embodiment, human antibodies of the disclosure can be raised using a mouse that carries human immunoglobulin sequences on transgenes and transchomosomes such as a mouse that carries a human heavy chain transgene and a human light chain transchromosome. Such mice, referred to herein as “KM mice”, are described in detail in PCT Publication WO 02/43478 to Ishida et al.

Still further, alternative transgenic animal systems expressing human immunoglobulin genes are available in the art and can be used to raise antibodies of the disclosure. For example, an alternative transgenic system referred to as the Xenomouse from Abgenix, Inc. can be used. Such mice are described in, e.g., U.S. Pat. Nos. 5,939,598; 6,075,181; 6,114,598; 6, 150,584 and 6,162,963 to Kucherlapati et al. As will be appreciated by a person skilled in the art, several other mouse models may be used, such as the TRIANNI mouse from Trianni, Inc., the VELOCIMMUNE mouse from Regeneron Pharmaceuticals, Inc., or the KYMOUSE mouse from Kymab Limited.

Moreover, alternative transchromosomic animal systems expressing human immunoglobulin genes are available in the art and can be used to raise anti-IL-17A antibodies of the disclosure. For example, mice carrying both a human heavy chain transchromosome and a human light chain transchromosome, referred to as “TC mice” can be used; such mice are described in Tomizuka et al. 2000, Proc. Natl. Acad. Sci. USA 97:722-727.

Human monoclonal antibodies of the disclosure can also be prepared using SCID mice into which human immune cells have been reconstituted such that a human antibody response can be generated upon immunization. Such mice are described in, for example, U.S. Pat. Nos. 5,476,996 and 5,698,767 to Wilson et al.

### Generation of Monoclonal Antibodies from a Murine System

Monoclonal antibodies (mAbs) can be produced by a variety of techniques, including conventional monoclonal antibody methodology e.g., the standard somatic cell hybridization technique of Kohler and Milstein 1975, Nature 256:495. Many techniques for producing monoclonal antibody can be employed e.g., viral or oncogenic transformation of B lymphocytes.

An animal system for preparing hybridomas is the murine system. Hybridoma production in the mouse is a well-established procedure. Immunization protocols and techniques for isolation of immunized splenocytes for fusion are known in the art. Fusion partners (e.g., murine myeloma cells) and fusion procedures are also known.

Chimeric or humanized antibodies of the present disclosure can be prepared based on the sequence of a murine monoclonal antibody prepared as described above. DNA encoding the heavy and light chain immunoglobulins can be obtained from the murine hybridoma of interest and engineered to contain non-murine (e.g., human) immunoglobulin sequences using standard molecular biology techniques. For example, to create a chimeric antibody, the murine variable regions can be linked to human constant regions using methods known in the art (see e.g., U.S. Pat. No. 4,816,567 to Cabilly et al.). To create a humanized antibody, the murine CDR regions can be inserted into a human framework using methods known in the art. See e.g., U.S. Pat. No. 5,225,539 to Winter, and U.S. Pat. Nos. 5,585,089; 5,693,762 and 6,180,370 to Queen et al.

### Generation of Hybridomas Producing Monoclonal Antibodies

To generate hybridomas producing monoclonal antibodies of the disclosure, splenocytes and/or lymph node cells from immunized mice can be isolated and fused to an appropriate immortalized cell line, such as a mouse myeloma cell line. The resulting hybridomas can be screened for the production of antigen-specific or epitope-specific antibodies. For example, single cell suspensions of splenic lymphocytes from immunized mice can be fused to one-sixth the number of P3X63-Ag8.653 nonsecreting mouse myeloma cells (ATCC, CRL 1580) with 50% PEG. Cells are plated at approximately 2×145 in flat bottom microtiter plates, followed by a two week incubation in selective medium containing 20% fetal Clone Serum, 18% “653” conditioned media, 5% origen (IGEN), 4mM L-glutamine, 1 mM sodium pyruvate, 5 mM HEPES, 0:055 mM 2-mercaptoethanol, 50 units/ml penicillin, 50 mg/ml streptomycin, 50 mg/ml gentamycin and 1× HAT (Sigma; the HAT is added 24 hours after the fusion). After approximately two weeks, cells can be cultured in medium in which the HAT is replaced with HT. Individual wells can then be screened by ELISA for human monoclonal IgM and IgG antibodies. Once extensive hybridoma growth occurs, medium can be observed usually after 10-14 days. The antibody secreting hybridomas can be replated, screened again, and if still positive for human IgG, the monoclonal antibodies can be subcloned once or twice by limiting dilution. The stable subclones can then be cultured in vitro to generate small amounts of antibody in tissue culture medium for characterization.

To purify monoclonal antibodies, selected hybridomas can be grown in two-liter spinner-flasks for monoclonal antibody purification. Supernatants can be filtered and concentrated before affinity chromatography with protein A-sepharose (Pharmacia, Piscataway, N.J.). Eluted IgG can be checked by gel electrophoresis and high performance liquid chromatography to ensure purity. The buffer solution can be exchanged into PBS, and the concentration can be determined by OD28o using 1.43 extinction coefficient. The monoclonal antibodies can be aliquoted and stored at −80° C.

### Generation of Transfectomas Producing Monoclonal Antibodies

Antibodies of the disclosure can be produced in a host cell transfectoma using, for example, a combination of recombinant DNA techniques and gene transfection methods as is well known in the art (e.g., Morrison, S. 1985, Science 229:1202).

For example, to express the antibodies, or antibody fragments thereof, DNAs encoding partial or full-length light and heavy chains can be obtained by standard molecular biology or biochemistry techniques (e.g., DNA chemical synthesis, PCR amplification or cDNA cloning using a hybridoma that expresses the antibody of interest) and the DNAs can be inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). The light and heavy chain variable regions of the antibodies described herein can be used to create full-length antibody genes of any antibody isotype by inserting them into expression vectors already encoding heavy chain constant and light chain constant regions of the desired isotype such that the Vu segment is operatively linked to the CH segment(s) within the vector and the V_{L} segment is operatively linked to the CL segment within the vector. Additionally or alternatively, the recombinant expression vector can encode a signal peptide, also called leader sequence, which facilitates secretion of the antibody chain from a host cell. The antibody chain gene can be cloned into the vector such that the signal peptide is linked in frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the disclosure carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel 1990, Gene Expression Technology. Methods in Enzymology 185, Academic Press, San Diego, CA). It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1 (Takebe, Y. et al. 1988, Mol. Cell. Biol. 8:466-472).

In addition to the antibody chain genes and regulatory sequences, the recombinant expression vectors of the disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Pat. Nos. 4,399,216, 4,634,665 and all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr-host cells with methotrexate selection/amplification) and the neo gene (for G418 selection).

For expression of the light and heavy chains, standard techniques were applied to transfect a host cell with the expression vector(s) encoding the heavy and light chains. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the antibodies of the disclosure in either prokaryotic or eukaryotic host cells. Expression of antibodies in eukaryotic cells, for example mammalian host cells, yeast or filamentous fungi, is discussed because such eukaryotic cells, and in particular mammalian cells, are more likely than prokaryotic cells to assemble and secrete a properly folded and immunologically active antibody.

In one specific embodiment, a cloning or expression vector according to the disclosure comprises at least one of the nucleic acid coding sequences of the disclosure, operatively linked to suitable promoter sequences.

Mammalian host cells for expressing the recombinant antibodies of the disclosure include Chinese Hamster Ovary (CHO cells) (including dhfr- CHO cells, described Urlaub and Chasin 1980, Proc. Natl. Acad. Sci. USA 77:4216-4220 used with a DH FR selectable marker, e.g., as described in R. J. Kaufman and P. A. Sharp 1982, Mol. Biol. 159:601-621), CHOK1 dhfr+ cell lines, NSO myeloma cells, COS cells and SP2 cells. In particular, for use with NSO myeloma cells, another expression system is the GS gene expression system shown in PCT Publications WO 87/04462, WO 89/01036 and EP 0 338 841. In one embodiment, mammalian host cells for expressing the recombinant antibodies of the disclosure include mammalian cell lines deficient for FUT8 gene expression, for example as described in U.S. Pat. No. 6,946,292.

When recombinant expression vectors encoding antibody genes are introduced into mammalian host cells, the antibodies are produced by culturing the host cells for a period of time sufficient to allow for expression of the antibody in the host cells or secretion of the antibody into the culture medium in which the host cells are grown. Antibodies can be recovered from the culture medium using standard protein purification methods (See for example Abhinav et al. 2007, Journal of Chromatography 848:28-37).

In one embodiment, the host cell of the disclosure is a host cell transfected with an expression vector(s) having nucleic acid(s) encoding an antibody or antigen-binding fragment of the disclosure.

These host cells may then be further cultured under suitable conditions for the expression and production of an antibody or antigen-binding fragment of the disclosure.

### Multispecific Molecules

In another aspect, the present disclosure features bispecific or multispecific molecules comprising a binding molecule, for example, an antibody, comprising a modified nd asilenced IgG1 Fc fragement of the disclosure. An antibody or protein of the disclosure can be derivatized or linked to another functional molecule, e.g., another peptide or protein (e.g., another antibody or ligand for a receptor) to generate a bispecific molecule that binds to at least two different binding sites or target molecules. The antibody or protein of the disclosure may in fact be derivatized or linked to more than one other functional molecule to generate multi-specific molecules that bind to more than two different binding sites and/or target molecules; such multi-specific molecules are also intended to be encompassed by the term “bispecific molecule” as used herein. To create a bispecific molecule of the disclosure, an antibody or protein of the disclosure can be functionally linked (e.g., by chemical coupling, genetic fusion, noncovalent association or otherwise) to one or more other binding molecules, such as another antibody, antibody fragment, peptide or binding mimetic, such that a bispecific molecule results. Methods for generating bispecific antibodies are well known in the art, for example, as described in Krah et al, 2017, New Biotechnology, 2017, 39:167-173; Brinkmann and Kontermann, 2017, Mabs, 9(2):182-212; Godar et al., 2018, Expert Opinion on Therapeutic Patents, 28(3):251-256; Spiess et al., 2015, Molecular Immunology 67:95-106; and Kontermann and Brinkmann, 2015, 20(7):838-847.

Additionally, for the disclosure in which the bispecific molecule is multi-specific, the molecule can further include a third binding specificity, in addition to the first and second target epitope.

In one embodiment, the silenced IgG1 Fc containing bispecific or multispecific molecules of the disclosure comprise as a binding specificity at least one antibody, or an antibody fragment thereof, including, e.g., an Fab, Fab′, F(ab′)2, Fv, or scFv. The antibody may also be a light chain or heavy chain dimer, or any minimal fragment thereof such as a Fv or a single chain construct as described in Ladner et al. U.S. Pat. No. 4,946,778.

Other antibodies which can be employed in the bispecific or multispecific molecules of the disclosure are murine, chimeric and humanized monoclonal antibodies.

The silenced IgG1 Fc containing bispecific or multispecific molecules of the present disclosure can be prepared by conjugating the constituent binding specificities, using methods known in the art. For example, each binding-specificity of the bispecific molecule can be generated separately and then conjugated to one another. When the binding specificities are proteins or peptides, a variety of coupling or cross-linking agents can be used for covalent conjugation. Examples of cross-linking agents include protein A, carbodiimide, N-succinimidyl-S-acetyl-thioacetate (SATA), 5,5′-dithiobis(2-nitrobenzoic acid) (DTNB), o-phenylenedimaleimide (oPDM), N-succinimidyl-3-(2-pyridyldithio)propionate (SPDP), and sulfosuccinimidyl 4-(N-maleimidomethyl) cyclohaxane-1-carboxylate (sulfo-SMCC) (see e.g., Karpovsky et al. 1984, J. Exp. Med. 160:1686; Liu, MA et al. 1985, Proc. Natl. Acad. Sci. USA 82:8648). Other methods include those described in Paulus 1985, Behring Ins. Mitt. No. 78,118-132; Brennan et al. 1985, Science 229:81-83), and Glennie et al. 1987, J. Immunol. 139: 2367-2375). Conjugating agents are SATA and sulfo-SMCC, both available from Pierce Chemical Co. (Rockford, IL).

When the binding specificities are antibodies, they can be conjugated by sulfhydryl bonding of the C-terminus hinge regions of the two heavy chains. In a particular embodiment, the hinge region is modified to contain an odd number of sulfhydryl residues, for example one, prior to conjugation.

The binding specificities can be encoded in the same vector and expressed and assembled in the same host cell. This method is particularly useful where the bispecific molecule is a mAb x mAb, mAb x Fab, Fab x F(ab′)2 or ligand x Fab fusion protein. A bispecific or multispecific molecule of the disclosure can be a single chain molecule comprising one single chain antibody and a binding determinant, or a single chain multispecific molecule comprising two binding determinants. Multispecific molecules may comprise at least two single chain molecules. Methods for preparing multispecific molecules are described for example in U.S. Pat. Nos. 5,260,203; 5,455,030; 4,881,175; 5,132,405; 5,091,513; 5,476,786; 5,013,653; 5,258,498; and 5,482,858.

Binding of the multispecific molecules to their specific targets can be confirmed by, for example, enzyme-linked immunosorbent assay (ELISA), radioimmunoassay (REA), FACS analysis, bioassay (e.g., growth inhibition), or Western Blot assay. Each of these assays generally detects the presence of protein-antibody complexes of particular interest by employing a labeled reagent (e.g., an antibody) specific for the complex of interest.

### Knob-in-Hole (KIH) (also known as “Key-in-Hole”)

Multispecific silenced IgG1 Fc containing binding molecules, e.g., multi-specific antibody or antibody-like molecules of the present invention may comprise one or more, e.g., a plurality, of mutations to one or more of the constant domains, e.g., to the CH3 domains. In one example, the multi-specific binding molecule of the present invention comprises two polypeptides that each comprise a heavy chain Fc or a constant domain of an antibody, e.g., a CH2 or CH3 domain. In an example, the two heavy chain constant domains, e.g., the CH2 or CH3 domains of the multi-specific binding molecule, comprise one or more mutations that allow for a heterodimeric association between the two chains. In one aspect, the one or more mutations are disposed on the CH2 domain of the two heavy chains of the multi-specific, e.g., bispecific, antibody or antibody-like molecule. In one aspect, the one or more mutations are disposed on the CH3 domains of at least two polypeptides of the multi-specific binding molecule. In one aspect, the one or more mutations to a first polypeptide of the multi-specific binding molecule comprising a heavy chain constant domain creates a “knob” and the one or more mutations to a second polypeptide of the multi-specific binding molecule comprising a heavy chain constant domain creates a “hole,” such that heterodimerization of the polypeptide of the multi-specific binding molecule comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” As the term is used herein, a “knob” refers to at least one amino acid side chain which projects from the interface of a first polypeptide of the multi-specific binding molecule comprising a heavy chain constant domain and is therefore positionable in a compensatory “hole” in the interface with a second polypeptide of the multi-specific binding molecule comprising a heavy chain constant domain so as to stabilize the heteromultimer, and thereby favor heteromultimer formation over homomultimer formation, for example. The knob may exist in the original interface or may be introduced synthetically (e.g., by altering nucleic acid encoding the interface). The preferred import residues for the formation of a knob are generally naturally occurring amino acid residues and are preferably selected from arginine (R), phenylalanine (F), tyrosine (Y) and tryptophan (W). Most preferred are tryptophan and tyrosine. In the preferred embodiment, the original residue for the formation of the protuberance has a small side chain volume, such as alanine, asparagine, aspartic acid, glycine, serine, threonine or valine.

A “hole” refers to at least one amino acid side chain which is recessed from the interface of a second polypeptide of the multi-specific binding molecule comprising a heavy chain constant domain and therefore accommodates a corresponding knob on the adjacent interfacing surface of a first polypeptide of the multi-specific binding molecule comprising a heavy chain constant domain. The hole may exist in the original interface or may be introduced synthetically (e.g., by altering nucleic acid encoding the interface). The preferred import residues for the formation of a hole are usually naturally occurring amino acid residues and are preferably selected from alanine (A), serine (S), threonine (T) and valine (V). Most preferred are serine, alanine or threonine. In the preferred embodiment, the original residue for the formation of the hole has a large side chain volume, such as tyrosine, arginine, phenylalanine or tryptophan.

In one embodiment, a first CH3 domain is mutated at residue 366, 405 or 407 according to the EU numbering scheme of Kabat et al. (pp. 688-696 in Sequences of proteins of immunological interest, 5th ed., Vol. 1 (1991; NIH, Bethesda, Md.)) to create either a “knob” or a hole” (as described above), and the second CH3 domain that heterodimerizes with the first CH3 domain is mutated at: residue 407 if residue 366 is mutated in the first CH3 domain, residue 394 if residue 405 is mutated in the first CH3 domain, or residue 366 if residue 407 is mutated in the first CH3 domain, according to the EU numbering scheme of Kabat et al. (pp. 688-696 in Sequences of proteins of immunological interest, 5th ed., Vol. 1 (1991; NIH, Bethesda, Md.)), to create a “hole” or “knob” complementary to the “knob” or “hole” of the first CH3 domain.

In another embodiment, a first CH3 domain is mutated at residue 366 according to the EU numbering scheme of Kabat et al. (pp. 688-696 in Sequences of proteins of immunological interest, 5th ed., Vol. 1 (1991; NIH, Bethesda, Md.)) to create either a “knob” or a hole” (as described above), and the second CH3 domain that heterodimerizes with the first CH3 domain is mutated at residues 366, 368 and/or 407, according to the EU numbering scheme of Kabat et al. (pp. 688-696 in Sequences of proteins of immunological interest, 5th ed., Vol. 1 (1991; NIH, Bethesda, Md.)), to create a “hole” or “knob” complementary to the “knob” or “hole” of the first CH3 domain. In one embodiment, the mutation to the first CH3 domain introduces a tyrosine (Y) residue at position 366. In an embodiment, the mutation to the first CH3 is T366Y. In one embodiment, the mutation to the first CH3 domain introduces a tryptophan (W) residue at position 366. In an embodiment, the mutation to the first CH3 is T366W. In embodiments, the mutation to the second CH3 domain that heterodimerizes with the first CH3 domain mutated at position 366 (e.g., has a tyrosine (Y) or tryptophan (W) introduced at position 366, e.g., comprises the mutation T366Y or T366W), comprises a mutation at position 366, a mutation at position 368 and a mutation at position 407, according to the EU numbering scheme of Kabat et al. (pp. 688-696 in Sequences of proteins of immunological interest, 5th ed., Vol. 1 (1991; NIH, Bethesda, Md.)) In embodiments, the mutation at position 366 introduces a serine (S) residue, the mutation at position 368 introduces an alanine (A), and the mutation at position 407 introduces a valine (V). In embodiments, the mutations comprise T366S, L368A and Y407V. In one embodiment the first CH3 domain of the multi-specific binding molecule comprises the mutation T366Y, and the second CH3 domain that heterodimerizes with the first CH3 domain comprises the mutations T366S, L368A and Y407V, or vice versa. In one embodiment the first CH3 domain of the multi-specific binding molecule comprises the mutation T366W, and the second CH3 domain that heterodimerizes with the first CH3 domain comprises the mutations T366S, L368A and Y407V, or vice versa.

Additional knob in hole mutation pairs suitable for use in any of the multi-specific binding molecules of the present invention are further described in, for example, WO1996/027011, and Merchant et al., Nat. Biotechnol., 16:677-681 (1998), the contents of which are hereby incorporated by reference in their entirety.

In any of the embodiments described herein, the CH3 domains may be additionally mutated to introduce a pair of cysteine residues. Without being bound by theory, it is believed that the introduction of a pair of cysteine residues capable of forming a disulfide bond provide stability to the heterodimerized multi-specific binding molecule. In embodiments, the first CH3 domain comprises a cysteine at position 354, according to the EU numbering scheme of Kabat et al. (pp. 688-696 in Sequences of proteins of immunological interest, 5th ed., Vol. 1 (1991; NIH, Bethesda, Md.)), and the second CH3 domain that heterodimerizes with the first CH3 domain comprises a cysteine at position 349, according to the EU numbering scheme of Kabat et al. (pp. 688-696 in Sequences of proteins of immunological interest, 5th ed., Vol. 1 (1991; NIH, Bethesda, Md.)) In embodiments, the first CH3 domain of the multi-specific binding molecule comprises a cysteine at position 354 (e.g., comprises the mutation S354C) and a tyrosine (Y) at position 366 (e.g., comprises the mutation T366Y), and the second CH3 domain that heterodimerizes with the first CH3 domain comprises a cysteine at position 349 (e.g., comprises the mutation Y349C), a serine at position 366 (e.g., comprises the mutation T366S), an alanine at position 368 (e.g., comprises the mutation L368A), and a valine at position 407 (e.g., comprises the mutation Y407V). In embodiments, the first CH3 domain of the multi-specific binding molecule comprises a cysteine at position 354 (e.g., comprises the mutation S354C) and a tryptophan (W) at position 366 (e.g., comprises the mutation T366W), and the second CH3 domain that heterodimerizes with the first CH3 domain comprises a cysteine at position 349 (e.g., comprises the mutation Y349C), a serine at position 366 (e.g., comprises the mutation T366S), an alanine at position 368 (e.g., comprises the mutation L368A), and a valine at position 407 (e.g., comprises the mutation Y407V).

An additional mechanism that finds use in the generation of heterodimers is sometimes referred to as “electrostatic steering” as described in Gunasekaran et al., 2010, J. Biol. Chem. 285(25):19637 and Strop et al., 2012, J. Mol. Biol. 420:204-19. This is sometimes referred to herein as “charge pairs”. In this embodiment, electrostatics are used to skew the formation towards heterodimerization. Two positively charged lysines (D339K, E356K) onto one chain and two negatively charged aspartates onto the other (K409D, K392D). In another embodiment, mutations are introduced not only on the CH3 domain (chainA: L368E, chain B:K409D), but also the hinge region (chain A:D221E and P228E, chain B:D221R and P228R) of IgG1. In another embodiment, the mutations include D221E/P228E/L368E paired with D221R/P228R/K409R and C220E/P228E/368E paired with C220R/E224R/P228R/K409R.

In certain embodiments, multi-specific (e.g., a bispecific or a trispecific) antibody or antibody-like molecule is also generated by Fab arm exchange as described in, Labrijin et al., 2011, J. Immunol. 187:3239-46; Labrijin et al., 2013, Proc. Natl. Acad. Sci., 110:5145-50, WO 08/119353, WO 2011/131746, and WO 2013/060867. In another embodiment, the multi-specific antibody includes mutations in the CH3 region that promotes Fc heterodimerisation: S364H, Y349T and T394H, and optionally an additional stability enhacing mutation T350V) as described in Moore et al., 2011, Mabs, 3:546-57.

In certain embodiments, the multi-specific (e.g., a bispecific or a trispecific) antibody or antibody-like molecule is generated by Strand Exchange Engineered Domains (SEED) heterodimer formation as described in, e.g., Davis et al., 2010, Protein Eng. Des. Sel., 23:195-202; Muda et al., 2011, Protein Eng. Des. Sel., 24:447-454; and WO 07/110205. In these embodiments, substantial changes are introduced into the Fc region by engineering of alternative IgG and IgA segments in the CH3 domains, resulting in two non-identical, anti-parallel chains, designated GA and AG, which buid up an asymmetric heterodimerisation interface.

In certain embodiments, the multi-specific (e.g., a bispecific or a trispecific) antibody or antibody-like molecule is generated by other technologies well known in the art, including double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody or antibody-like molecule determinants generated by recombining half antibodies (heavy-light chain pairs or Fabs) from different antibodies or antibody-like molecules through cycle of reduction and oxidation of disulfide bonds between the two heavy chains, as described in, e.g., U.S. Pat. No. 4,444,878; trifunctional antibodies, e.g., three Fab′ fragments cross-linked through sulfhdryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono-and oligovalent receptors, e.g., V_{H}-CH1 regions (Fd regions) of two antibodies (two Fab fragments) linked through a polypeptide spacer between the CH1 region of one antibody and the V_{H} region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multi-specific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked V_{L} and V_{H} chains further connected with peptide spacers to an antibody hinge region and CH3 region, which can be dimerized to form bispecific/multivalent molecules, as described in, e.g., U.S. Pat. No. 5,837,821; V_{L} and V_{H} domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; string of V_{H} domains (or V_{L} domains in family members) connected by peptide linkages with crosslinkable groups at the C-terminus further associated with V_{L} domains to form a series of FVs (or scFvs), as described in, e.g., U.S. Pat. No. 5,864,019; V_{L} and V_{H} domains, scFvs, or Fabs wherein one of the antigens is bound monovalently and one of the antigens is bound bivalently, optionally comprising heterodimeric Fc regions, as described in, e.g., WO2011/028952; and single chain binding polypeptides with both a V_{L} and V_{H} domain linked through a peptide linker are combined into multivalent structures through non-covalent or chemical crosslinking to form, e.g., homobivalent, heterobivalent, trivalent, and tetravalent structures using both scFv or diabody type format, as described in, e.g., U.S. Pat. No. 5,869,620. Additional exemplary multi-specific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

In another aspect, the present disclosure provides modified IgG1 Fc containing multivalent and multispecific antibodies comprising at least two identical or different antigen-binding portions of the antibodies of the disclosure. In one embodiment, the multivalent antibodies provide at least two, three or four antigen-binding portions of the antibodies. The antigen-binding portions can be linked together via protein fusion or covalent or non-covalent linkage. Alternatively, methods of linkage have been described for the bispecific molecules. Tetravalent compounds can be obtained for example by cross-linking antibodies of the disclosure with an antibody that binds to the constant regions of the antibodies of the disclosure, for example the Fc or hinge region.

### Therapeutic Methods

In one aspect, the modified IgG1 Fc containing binding molecules according to the invention are used for treating a disease. In a more specific aspect, the disease is such, that it is favorable that the effector function of the variant is strongly reduced by at least by 50%, 70%, 80%, 90%, 95%, 98% or 99% reduced or undetectable, as compared to a polypeptide comprising the wildtype IgG Fc polypeptide.

In a specific aspect the modified IgG1 Fc containing binding molecule according to the invention is for use as a medicament. Preferred are uses wherein it is favorable that the effector function of the polypeptide is strongly reduced compared to a wildtype Fc polypeptide. In a further specific aspect the binding molecule according to the invention is for use as a medicament, for use in treatment of a disease, wherein it is favorable that the effector function of the polypeptide is reduced compared to a wildtype Fc polypeptide, by at least 50%,70%, 80%, 90%, 95%, 98% or 99% or undetectable.

A further aspect is a method of treating an individual having a disease, wherein it is favorable that the effector function of the variant is strongly reduced compared to a wildtype IgG1 Fc containing binding molecule, comprising administering to the individual an effective amount of the binding molecule according to the invention.

A strong reduction of effector function is a reduction of effector function by at least 50%, 70%, 80%, 90%, 95%, 98% or 99% or undetectable effector function as compared to effector functions induced by the wildtype polypeptide.

Such diseases are for example all diseases where the targeted antigen by the modified IgG1 Fc containing binding molecule can be present on a cell surface and the cell should not be destroyed by for example ADCC, ADCP and/or CDC, or diseases treatable with therapeutic antibodies or binding molecules that are designed to deliver a drug (e.g., toxins and radio-isotopes) to the target cell where the Fc/FcγR mediated effector functions bring healthy immune cells into the proximity of the deadly payload, resulting in depletion of normal lymphoid tissue along with the target cells (Hutchins, et al, PNAS USA 92 (1995) 11980-11984; White, et al, Annu Rev Med 52 (2001) 125-145). In these cases the use of antibodies that poorly recruit complement or effector cells would be of tremendous benefit (see for example, Wu, et al., Cell Immunol 200 (2000) 16-26; Shields, et al., J. Biol Chem 276(9) (2001) 6591-6604; U.S. Pat. Nos. 6,194,551; 5,885,573 and PCT publication WO 04/029207).

In other instances diseases, for example, where blocking the interaction of a widely expressed receptor with its cognate ligand is the objective of therapy, it would be advantageous to decrease or eliminate all antibody effector function to reduce unwanted toxicity. Also, in the instance where a therapeutic antibody exhibited promiscuous binding across a number of human tissues and or/cell surfaces, it would be prudent to limit the targeting of effector function to a diverse set of tissues and cells to limit toxicity.

### Pharmaceutical Compositions

Disclosed herein are pharmaceutical compositions comprising modified IgG1 Fc containing binding molecule as described herein, in combination with one or more pharmaceutically acceptable excipient, diluent or carrier.

Disclosed herein are pharmaceutical compositions comprising binding molecule as described herein, in combination with one or more additional therapeutic agent.

The term “pharmaceutical composition” refers to a mixture of at least one active ingredient (e.g., an antibody or fragment of the disclosure) and at least one pharmaceutically-acceptable excipient, diluent or carrier.

A “medicament” refers to a substance used for medical treatment.

The phrase “pharmaceutically acceptable” means approved by a regulatory agency of a federal or a state government, or listed in the U.S. Pharmacopeia or other generally recognized pharmacopeia for use in animals, and more particularly, in humans.

Pharmaceutically acceptable carriers includes any and all solvents, dispersion media, coatings, antibacterial and antifungal agents, isotonic and absorption delaying agents, and the like that are physiologically compatible. The carrier should be suitable for intravenous, intramuscular, subcutaneous, parenteral, spinal or epidermal administration (e.g., by injection or infusion). In one embodiment, the carrier should be suitable for subcutaneous route. Depending on the route of administration, the active compound, i.e., antibody, immunoconjugate, or bispecific molecule, may be coated in a material to protect the compound from the action of acids and other natural conditions that may inactivate the compound.

Formulations of therapeutic and diagnostic agents can be prepared by mixing with physiologically acceptable carriers, excipients, or stabilizers in the form of, e.g., lyophilized powders, slurries, aqueous solutions, lotions, or suspensions (see, e.g., Hardman et al., Goodman and Gilman's The Pharmacological Basis of Therapeutics, McGraw-Hill, New York, N.Y., 2001; Gennaro, Remington: The Science and Practice of Pharmacy, Lippincott, Williams, and Wilkins, New York, N.Y., 2000; Avis, et al. (eds.), Pharmaceutical Dosage Forms: Parenteral Medications, Marcel Dekker, NY, 1993; Lieberman, et al. (eds.), Pharmaceutical Dosage Forms: Tablets, Marcel Dekker, NY, 1990; Lieberman, et al. (eds.) Pharmaceutical Dosage Forms: Disperse Systems, Marcel Dekker, NY, 1990; Weiner and Kotkoskie, Excipient Toxicity and Safety, Marcel Dekker, Inc., New York, N.Y., 2000).

The binding molecules of the disclosure may be produced as a lyophilisate in a vial. The lyophilisate can be reconstituted with water or a pharmaceutical carrier suitable for injection. For subsequent intravenous administration, the obtained solution will usually be further diluted into a carrier solution.

Selecting an administration regimen for a therapeutic depends on several factors, including the severity of the infection, the level of symptoms, and the accessibility of the target cells in the biological matrix. In certain aspects, an administration regimen maximizes the amount of therapeutic delivered to the patient consistent with an acceptable level of side effects. Accordingly, the amount of biologic delivered depends in part on the particular entity and the severity of the condition being treated. Guidance in selecting appropriate doses of antibodies, cytokines, and small molecules are available (see, e.g., Wawrzynczak, Antibody Therapy, Bios Scientific Pub. Ltd, Oxfordshire, UK, 1996; Kresina (ed.), Monoclonal Antibodies, Cytokines and Arthritis, Marcel Dekker, New York, N.Y., 1991; Bach (ed.), Monoclonal Antibodies and Peptide Therapy in Autoimmune Diseases, Marcel Dekker, New York, N.Y., 1993; Baert et al., New Engl. J. Med. 348:601-608, 2003; Milgrom et al., New Engl. J. Med. 341:1966-1973, 1999; Slamon et al., New Engl. J. Med. 344:783-792, 2001; Beniaminovitz et al., New Engl. J. Med. 342:613-619, 2000; Ghosh et al., New Engl. J. Med. 348:24-32, 2003; Lipsky et al., New Engl. J. Med. 343:1594-1602, 2000).

Determination of the appropriate dose is made by the clinician, e.g., using parameters or factors known or suspected in the art to affect treatment or predicted to affect treatment. Generally, the dose begins with an amount somewhat less than the optimum dose and it is increased by small increments thereafter until the desired or optimum effect is achieved relative to any negative side effects. Important diagnostic measures include those of symptoms of, e.g., infusion reactions.

Actual dosage levels of the active ingredients in the pharmaceutical compositions with the binding molecule can be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the antibodies, the route of administration, the time of administration, the half-life of the antibody in the patient, the duration of the treatment, other drugs, compounds and/or materials used in combination with the particular compositions employed, the age, sex, weight, condition, general health and prior medical history of the patient being treated, and like factors known in the medical arts.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of sensitivity in individuals.

Compositions comprising antibodies or fragments thereof can be provided by continuous infusion, or by doses at intervals of, e.g., one day, one week, or 1-7 times per week. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

For administration of the antibody or protein, the dosage ranges from about to 150 mg/kg, such as 5, 15, and 50 mg/kg subcutaneous administration, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once per month, once every 3 months or once every three to 6 months. Dosage regimens for an antibody or fragment of the disclosure include 1 mg/kg body weight, 3 mg/kg body weight, 5 mg/kg, 10 mg/kg, 20 mg/kg or 30 mg/kg by intravenous administration, with the antibody being given using one of the following dosing schedules: every four weeks for six dosages, then every three months; every three weeks; 3 mg/kg body weight once followed by 1 mg/kg body weight every three weeks. Doses of the antibodies then can be repeated and the administrations may be separated by at least 1 day, 2 days, 3 days, 5 days, 10 days, 15 days, 30 days, 45 days, 2 months, 75 days, 3 months, or at least 6 months.

An effective amount for a particular patient may vary depending on factors such as the condition being treated, the overall health of the patient, the method, route and dose of administration and the severity of side effects (see, e.g., Maynard et al., A Handbook of SOPs for Good Clinical Practice, Interpharm Press, Boca Raton, Fla., 1996; Dent, Good Laboratory and Good Clinical Practice, Urch Publ., London, UK, 2001).

The route of administration may be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., Biopolymers 22:547-556, 1983; Langer et al., J. Biomed. Mater. Res. 1981; Langer, Chem. Tech. 12:98-105, 1982; Epstein et al., Proc. Natl. Acad. Sci. USA 82:3688-3692, 1985; Hwang et al., Proc. Natl. Acad. Sci. USA 77:4030-4034, 1980; U.S. Pat. Nos. 6,350,466 and 6,316,024). Where necessary, the composition may also include a solubilizing agent or a local anesthetic such as lidocaine to ease pain at the site of the injection, or both. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

A composition containing silenced, modified IgG1 Fc containing binding molecules and antibodies of the present invention can also be administered via one or more routes of administration using one or more of a variety of methods known in the art. As will be appreciated by the skilled artisan, the route and/or mode of administration will vary depending upon the desired results. Selected routes of administration for the antibodies include intravenous, intramuscular, intradermal, intraperitoneal, subcutaneous, spinal or other parenteral routes of administration, for example by injection or infusion. Parenteral administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the present disclosure can be administered via a non-parenteral route, such as a topical, epidermal or mucosal route of administration, for example, intranasally, orally, vaginally, rectally, sublingually or topically. In one aspect, the antibodies of the present disclosure are administered by infusion. In another aspect, the antibodies are administered subcutaneously.

If the modified IgG1 Fc containing binding molecules or antibodies of the present invention are administered in a controlled release or sustained release system, a pump may be used to achieve controlled or sustained release (see Langer, supra; Sefton, CRC Crit. Ref Biomed. Eng. 14:20, 1987; Buchwald et al., Surgery 88:507, 1980; Saudek et al., N. Engl. J. Med. 321:574, 1989). Polymeric materials can be used to achieve controlled or sustained release of the therapies of the antibodies (see e.g., Medical Applications of Controlled Release, Langer and Wise (eds.), CRC Pres., Boca Raton, Fla., 1974; Controlled Drug Bioavailability, Drug Product Design and Performance, Smolen and Ball (eds.), Wiley, New York, 1984; Ranger and Peppas, J. Macromol. Sci. Rev. Macromol. Chem. 23:61, 1983; see also Levy et al., Science 228:190, 1985; During et al., Ann. Neurol. 25:351, 1989; Howard et al., J. Neurosurg. 7 1:105, 1989; U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one aspect, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138, 1984).

Controlled release systems are discussed in the review by Langer, Science 249:1527-1533, 1990). Any technique known to one of skill in the art can be used to produce sustained release formulations comprising one or more antibodies of the present disclosure. See, e.g., U.S. Pat. No. 4,526,938, PCT publication WO 91/05548, PCT publication WO 96/20698, Ning et al., Radiotherapy & Oncology 39:179-189, 1996; Song et al., PDA Journal of Pharmaceutical Science & Technology 50:372-397, 1995; Cleek et al., Pro. Int'l. Symp. Control. Rel. Bioact. Mater. 24:853-854, 1997; and Lam et al., Proc. Int'l. Symp. Control Rel. Bioact. Mater. 24:759-760, 1997, each of which is incorporated herein by reference in their entirety.

Various means for administering therapeutic compositions are known in the art. For example, in one embodiment, a therapeutic composition of the disclosure can be administered with a needleless hypodermic injection device, such as the devices shown in U.S. Pat. Nos. 5,399,163; 5,383,851; 5,312,335; 5,064,413; 4,941,880; 4,790,824 or 4,596,556. Examples of well-known implants and modules useful in the present disclosure include: U.S. Pat. No. 4,487,603, which shows an implantable micro-infusion pump for dispensing medication at a controlled rate; U.S. Pat. No. 4,486,194, which shows a therapeutic device for administering medicants through the skin; U.S. Pat. No. 4,447,233, which shows a medication infusion pump for delivering medication at a precise infusion rate; U.S. Pat. No. 4,447,224, which shows a variable flow implantable infusion apparatus for continuous drug delivery; U.S. Pat. No. 4,439,196, which shows an osmotic drug delivery system having multi-chamber compartments; and U.S. Pat. No. 4,475,196, which shows an osmotic drug delivery system. Many other such implants, delivery systems, and modules are known to those skilled in the art. In preferred embodiments, the means for administering the antibodies and fragments is selected from a syringe, an autoinjector, an injection pen, a vial and syringe, an infusion pump, a patch, or an infusion bag and needle.

If the silenced IgG1 Fc containing binding molecules and antibodies of the invention are administered topically, they can be formulated in the form of an ointment, cream, transdermal patch, lotion, gel, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known in the art.

If the compositions comprising the modified, silenced IgG1 Fc containing binding molecules and antibodies of the invention are administered intranasally, it can be formulated in an aerosol form, spray, mist or in the form of drops. In particular, prophylactic or therapeutic agents for use according to the present disclosure can be conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

Methods for co-administration or treatment with an additional therapeutic agent, e.g., an immunosuppressant, a cytokine, steroid, chemotherapeutic agent, antibiotic, etc., are known in the art (see, e.g., Hardman et al., (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.). An effective amount of therapeutic may decrease the symptoms by at least 10%; by at least 20%; at least about 30%; at least 40%, or at least 50%.

Additional therapies (e.g., prophylactic or therapeutic agents), which can be administered in combination with the antibodies may be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart from the antibodies and fragments of the present disclosure. The two or more therapies may be administered within one same patient visit.

In certain aspects, the binding molecules of the disclosure can be formulated to ensure proper distribution in vivo. For example, the blood-brain barrier (BBB) excludes many highly hydrophilic compounds. To ensure that the binding molecules cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties, which are selectively transported into specific cells or organs, thus enhance targeted drug delivery (see, e.g., Ranade, (1989) J. Clin. Pharmacol. 29:685). Exemplary targeting moieties include folate or biotin (see, e.g., U.S. Pat. No. 5,416,016 to Low et al.); mannosides (Umezawa et al., (1988) Biochem. Biophys. Res. Commun. 153:1038); antibodies (Bloeman et al., (1995) FEBS Lett. 357:140; Owais et al., (1995) Antimicrob. Agents Chemother. 39:180); surfactant protein A receptor (Briscoe et al., (1995) Am. J. Physiol. 1233:134); p 120 (Schreier et al, (1994) J. Biol. Chem. 269:9090); see also K. Keinanen; M. L. Laukkanen (1994) FEBS Lett. 346:123; J. J. Killion; I. J. Fidler (1994) Immunomethods 4:273.

The present disclosure provides protocols for the administration of pharmaceutical composition comprising the modified and silenced IgG1 Fc comprising binding molecule alone or in combination with other therapies to a subject in need thereof. The combination therapies (e.g., prophylactic or therapeutic agents) can be administered concomitantly or sequentially to a subject. The therapy (e.g., prophylactic or therapeutic agents) of the combination therapies can also be cyclically administered. Cycling therapy involves the administration of a first therapy (e.g., a first prophylactic or therapeutic agent) for a period of time, followed by the administration of a second therapy (e.g., a second prophylactic or therapeutic agent) for a period of time and repeating this sequential administration, i.e., the cycle, in order to reduce the development of resistance to one of the therapies (e.g., agents) to avoid or reduce the side effects of one of the therapies (e.g., agents), and/or to improve, the efficacy of the therapies.

The therapies (e.g., prophylactic or therapeutic agents) of the combination therapies of the disclosure can be administered to a subject concurrently. The term “concurrently” is not limited to the administration of therapies (e.g., prophylactic or therapeutic agents) at exactly the same time, but rather it is meant that a pharmaceutical composition comprising antibodies or fragments thereof are administered to a subject in a sequence and within a time interval such that the antibodies can act together with the other therapy(ies) to provide an increased benefit than if they were administered otherwise. For example, each therapy may be administered to a subject at the same time or sequentially in any order at different points in time; however, if not administered at the same time, they should be administered sufficiently close in time so as to provide the desired therapeutic or prophylactic effect. Each therapy can be administered to a subject separately, in any appropriate form and by any suitable route. In various aspects, the therapies (e.g., prophylactic or therapeutic agents) are administered to a subject less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, 24 hours apart, 48 hours apart, 72 hours apart, or 1 week apart. In other aspects, two or more therapies (e.g., prophylactic or therapeutic agents) are administered to a within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference as applicable, unless otherwise indicated. The following Examples are presented in order to more fully illustrate the preferred embodiments of the disclosure. These examples should in no way be construed as limiting the scope of the disclosed subject matter, which is defined by the appended claims.

### EXAMPLES

### Example 1: Design—Selection of Residue Positions

Design strategies were tested to produce a set of antibodies with modified Fc regions that might exhibit desired properties such as diminished effector functions. Early studies defining key amino acid binding sites on IgG for Fc gamma receptors were performed by mutational analyses, and it was determined that the lower hinge, proximal CH2 region and glycosylation of N297 were critical (Shields et al., 2001). Mutations were introduced into the regions that interact with Fc gamma receptors with the goal to diminish residual binding to Fc gamma receptors. For this particular reason, it was necessary to test various combinations of Fc positions and generate set of mutations without compromising antibody drug developability and immunogenicity risk. Several mutation sets were generated and compared to wildtype IgG1. LALAPA-IgG1 (L234A/L235A/P329A), LALAGA-IgG1 (L234A/L235A/G237A), LALAPG-IgG1 (L234A/L235A/P329G), DAPA-IgG1 (D265A/P329A), LALASKPA-IgG1 (L234A/L235A/S267K/P329A), DAPASK-IgG1 (D265A/P329A/S267K), GADAPA-IgG1 (G237A/D265A/P329A), GADAPASK-IgG1 (G237A/D265A/P329A/S267K) and DANAPA-IgG1 (D265A/N297A/P329A) were evaluated. Previously described DAPA and DANAPA silencing motifs were included for comparison.

### Example 2: Expression and Purification of Modified Antibodies

For the experiments described below antibodies against CD3 (SEQ ID Nos: 1-24), containing the indicated amino acid substitutions and expressed by the nucleotide sequences as indicated, were used as listed in Table 1 below. IgG1 molecules were expressed in HEK293 mammalian cells, and purified using protein A and size exclusion chromatography. In brief, heavy chain and light chain DNA of anti-CD3 WT IgG1 were synthesized at GeneArt (Regensburg, Germany) and cloned into a mammalian expression vector using restriction enzyme-ligation based cloning techniques. All variants described herein were then generated using PCR based mutagenesis The resulting plasmids were co-transfected into HEK293T cells. For transient expression of antibodies, equal quantities of vector for each chain were co-transfected into suspension-adapted HEK293T cells using Polyethylenimine (PEI; Cat #24765 Polysciences, Inc.). Typically, 100 ml of cells in suspension at a density of 1-2 Mio cells per ml was transfected with DNA containing 50 μg of expression vector encoding the heavy chain and 50 μg expression vectors encoding the light chain. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 μg/ml antibiotic.

The produced construct was then purified from cell-free supernatant using immunoaffinity chromatography. Mab Select Sure resin (GE Healthcare Life Sciences), equilibrated with PBS buffer pH 7.4 was incubated with filtered conditioned media using liquid chromatography system (Aekta pure chromatography system, GE Healthcare Life Sciences). The resin was washed with PBS pH 7.4 before the constructs were eluted with elution buffer (50 mM citrate, 90 mM NaCl, pH 2.7). After capture, eluted proteins were pH neutralized using 1M TRIS pH 10.0 solution and polished using size exclusion chromatography technique (HiPrep Superdex 200 16/60, GE Healthcare Life Sciences). Purified proteins were finally formulated in PBS buffer pH 7.4.

TABLE 1
Sequences of Antibodies and Fc variant

| SEQ ID | | |
|---|---|---|
| No | Description | Sequence |
| 1 | Anti-CD3 human | evqlvesggglvqpggslklscaasgftfntyamnwvrqasg |
| | IgG1 Heavy Chain | kglewvgrirskynnyatyyadsvkdrftisrddskstlylq |
| | Amino acid | mnslktedtavyycvrhgnfgnsyvswfaywgqgtlvtvssa |
| | sequence | stkgpsvfplapsskstsggtaalgclvkdyfpepvtvswns |
| | | galtsgvhtfpavlqssglyslssvvtvpssslgtqtyicnv |
| | | nhkpsntkvdkrvepkscdkthtcppcpapellggpsvflfp |
| | | pkpkdtlmisrtpevtcvvvdvshedpevkfnwyvdgvevhn |
| | | aktkpreeqynstyrvvsvltvlhqdwlngkeykckvsnkal |
| | | papiektiskakgqprepqvytlppsrdeltknqvsltclvk |
| | | gfypsdiavewesngqpennykttppvldsdgsfflyskltv |
| | | dksrwqqgnvfscsvmhealhnhytqkslslspgk |
| 2 | Anti-CD3 human | gaagtgcagctggtggaatctggcggcggactggtgcagcct |
| | IgG1 Heavy Chain | ggcggatctctgaagctgagctgtgccgccagcggcttcacc |
| | Nucleic acid | ttcaacacctacgccatgaactgggtgcgccaggcctctggc |
| | sequence | aagggcctggaatggggggacggatcagaagcaagtacaac |
| | | aattacgccacctactacgccgacagcgtgaaggaccggttc |
| | | accatcagccgggacgacagcaagagcaccctgtacctgcag |
| | | atgaacagcctgaaaaccgaggacaccgccgtgtactactgc |
| | | gtgcggcacggcaacttcggcaacagctatgtgtcttggttt |
| | | gcctactggggccagggcaccctcgtgacagtgagctcagct |
| | | agcaccaagggccccagcgtgttccccctggcgcccagcagc |
| | | aagagcaccagcggcggcacagccgccctgggctgcctggtg |
| | | aaggactacttccccgagccagtgaccgtgtcctggaacagc |
| | | ggagccctgacctccggcgtgcacaccttccccgccgtgctg |
| | | cagagcagcggcctgtacagcctgagcagcgtggtgaccgtg |
| | | cccagcagcagcctgggcacccagacctacatctgcaacgtg |
| | | aaccacaagcccagcaacaccaaggtggacaagagagtggag |
| | | cccaagagctgcgacaagacccacacctgccccccctgtcct |
| | | gcccctgaactgctgggcggaccctccgtgttcctgttcccc |
| | | ccaaagcccaaggacaccctgatgatcagccggacccccgaa |
| | | gtgacctgcgtggtggtggacgtgtcccacgaggaccctgaa |
| | | gtgaagttcaattggtacgtggacggcgtggaagtgcacaac |
| | | gccaagaccaagcccagagaggaacagtacaacagcacctac |
| | | cgggtggtgtccgtgctgaccgtgctgcaccaggactggctg |
| | | aacggcaaagagtacaagtgcaaagtctccaacaaggccctg |
| | | cctgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 3 | Anti-CD3 human | evqlvesggglvqpggslklscaasgftfntyamnwvrqasg |
| | IgG1 | kglewvgrirskynnyatyyadsvkdrftisrddskstlylq |
| | R214K allotype | mnslktedtavyycvrhgnfgnsyvswfaywgqgtlvtvssa |
| | Heavy Chain | stkgpsvfplapsskstsggtaalgclvkdyfpepvtvswns |
| | Amino acid | galtsgvhtfpavlqssglyslssvvtvpssslgtqtyicnv |
| | sequence | nhkpsntkvdkkvepkscdkthtcppcpapellggpsvflfp |
| | | pkpkdtlmisrtpevtcvvvdvshedpevkfnwyvdgvevhn |
| | | aktkpreeqynstyrvvsvltvlhqdwlngkeykckvsnkal |
| | | papiektiskakgqprepqvytlppsrdeltknqvsltclvk |
| | | gfypsdiavewesngqpennykttppvldsdgsfflyskltv |
| | | dksrwqqgnvfscsvmhealhnhytqkslslspgk |
| 4 | Anti-CD3 human | gaagtgcagctggtggaatctggcggcggactggtgcagcct |
| | IgG1 | ggcggatctctgaagctgagctgtgccgccagcggcttcacc |
| | R214K allotype | ttcaacacctacgccatgaactgggtgcgccaggcctctggc |
| | Heavy Chain | aagggcctggaatgggtgggacggatcagaagcaagtacaac |
| | Nucleic acid | aattacgccacctactacgccgacagcgtgaaggaccggttc |
| | sequence | accatcagccgggacgacagcaagagcaccctgtacctgcag |
| | | atgaacagcctgaaaaccgaggacaccgccgtgtactactgc |
| | | gtgcggcacggcaacttcggcaacagctatgtgtcttggttt |
| | | gcctactggggccagggcaccctcgtgacagtgagctcagct |
| | | agcaccaagggccccagcgtgttccccctggcgcccagcagc |
| | | aagagcaccagcggcggcacagccgccctgggctgcctggtg |
| | | aaggactacttccccgagccagtgaccgtgtcctggaacagc |
| | | ggagccctgacctccggcgtgcacaccttccccgccgtgctg |
| | | cagagcagcggcctgtacagcctgagcagcgtggtgaccgtg |
| | | cccagcagcagcctgggcacccagacctacatctgcaacgtg |
| | | aaccacaagcccagcaacaccaaggtggacaagaaggtggag |
| | | cccaagagctgcgacaagacccacacctgccccccctgtcct |
| | | gcccctgaactgctgggcggaccctccgtgttcctgttcccc |
| | | ccaaagcccaaggacaccctgatgatcagccggacccccgaa |
| | | gtgacctgcgtggtggtggacgtgtcccacgaggaccctgaa |
| | | gtgaagttcaattggtacgtggacggcgtggaagtgcacaac |
| | | gccaagaccaagcccagagaggaacagtacaacagcacctac |
| | | cgggtggtgtccgtgctgaccgtgctgcaccaggactggctg |
| | | aacggcaaagagtacaagtgcaaagtctccaacaaggccctg |
| | | cctgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 5 | Anti-CD3 human | qavvtqepsltvspggtvtltcrsstgavttsnyanwvqqkp |
| | Light Chain | gqaprgliggtnkrapwtparfsgsllgdkaaltlsgaqped |
| | Amino acid | eaeyfcalwysnlwvfgggtkltvlgqpkaapsvtlfppsse |
| | sequence | elqankatlvclisdfypgavtvawkadsspvkagvetttps |
| | | kqsnnkyaassylsltpeqwkshrsyscqvthegstvektva |
| | | ptecs |
| 6 | Anti-CD3 human | caggctgtcgtgacccaggaacctagcctgaccgtgtctcct |
| | Light Chain | ggcggaaccgtgaccctgacctgtagatctagcacaggcgcc |
| | Nucleic acid | gtgaccaccagcaactacgccaattgggtgcagcagaagccc |
| | sequence | ggccaggctcctagaggactgatcggcggcaccaacaagaga |
| | | gccccttggacccctgccagattcagcggctctctgctggga |
| | | gataaggccgccctgacactgtctggcgcccagcctgaggat |
| | | gaggccgagtacttttgcgccctgtggtacagcaacctgtgg |
| | | gtgttcggcggaggcaccaagctgaccgtgctgggccagcct |
| | | aaggccgctccctccgtgaccctgttcccccccagctccgag |
| | | gaactgcaggccaacaaggccaccctggtgtgcctgatcagc |
| | | gacttctaccctggcgccgtgaccgtggcctggaaggccgac |
| | | agcagccccgtgaaggccggcgtggagacaaccacccccagc |
| | | aagcagagcaacaacaagtacgccgccagcagctacctgagc |
| | | ctgacccccgagcagtggaagagccacagaagctacagctgc |
| | | caggtcacccacgagggcagcaccgtggagaaaaccgtggcc |
| | | cccaccgagtgcagc |
| 7 | Human IgG1 Fc | apeaaggpsvflfppkpkdtlmisrtpevtcvvvdvshedpe |
| | variant | vkfnwyvdgvevhnaktkpreeqynstyrvvsvltvlhqdwl |
| | L234A/L235A/ | ngkeykckvsnkalaapiektiskakgqprepqvytlppsrd |
| | P329A (LALAPA) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 8 | Human IgG1 Fc | gcccctgaagccgccggcggaccctccgtgttcctgttcccc |
| | variant | ccaaagcccaaggacaccctgatgatcagccggacccccgaa |
| | L234A/L235A/ | gtgacctgcgtggtggtggacgtgtcccacgaggaccctgaa |
| | P329A (LALAPA) | gtgaagttcaattggtacgtggacggcgtggaagtgcacaac |
| | Nucleic acid | gccaagaccaagcccagagaggaacagtacaacagcacctac |
| | sequence | cgggtggtgtccgtgctgaccgtgctgcaccaggactggctg |
| | | aacggcaaagagtacaagtgcaaagtctccaacaaggccctg |
| | | gccgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 9 | Human IgG1 Fc | apeaagapsvflfppkpkdtlmisrtpevtcvvvdvshedpe |
| | variant | vkfnwyvdgvevhnaktkpreeqynstyrvvsvltvlhqdwl |
| | L234A/L235A/ | ngkeykckvsnkalpapiektiskakgqprepqvytlppsrd |
| | G237A (LALAGA) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 10 | Human IgG1 Fc | gcccctgaagccgccggcgccccctccgtgttcctgttcccc |
| | variant | ccaaagcccaaggacaccctgatgatcagccggacccccgaa |
| | L234A/L235A/ | gtgacctgcgtggtggtggacgtgtcccacgaggaccctgaa |
| | G237A (LALAGA) | gtgaagttcaattggtacgtggacggcgtggaagtgcacaac |
| | Nucleic acid | gccaagaccaagcccagagaggaacagtacaacagcacctac |
| | sequence | cgggtggtgtccgtgctgaccgtgctgcaccaggactggctg |
| | | aacggcaaagagtacaagtgcaaagtctccaacaaggccctg |
| | | cctgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 11 | Human IgG1 Fc | apeaaggpsvflfppkpkdtlmisrtpevtcvvvdvshedpe |
| | variant | vkfnwyvdgvevhnaktkpreeqynstyrvvsvltvlhqdwl |
| | L234A/L235A/ | ngkeykckvsnkalgapiektiskakgqprepqvytlppsrd |
| | P329G (LALAPG) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 12 | Human IgG1 Fc | gcccctgaagccgccggcggaccctccgtgttcctgttcccc |
| | variant | ccaaagcccaaggacaccctgatgatcagccggacccccgaa |
| | L234A/L235A/ | gtgacctgcgtggtggtggacgtgtcccacgaggaccctgaa |
| | P329G (LALAPG) | gtgaagttcaattggtacgtggacggcgtggaagtgcacaac |
| | Nucleic acid | gccaagaccaagcccagagaggaacagtacaacagcacctac |
| | sequence | cgggtggtgtccgtgctgaccgtgctgcaccaggactggctg |
| | | aacggcaaagagtacaagtgcaaagtctccaacaaggccctg |
| | | ggcgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 13 | Human IgG1 Fc | apellggpsvflfppkpkdtlmisrtpevtcvvvavshedpe |
| | variant | vkfnwyvdgvevhnaktkpreeqynstyrvvsvltvlhqdwl |
| | D265A/P329A | ngkeykckvsnkalaapiektiskakgqprepqvyt lppsrd |
| | (DAPA) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 14 | Human IgG1 Fc | gcccctgaactgctgggcggaccctccgtgttcctgttcccc |
| | variant | ccaaagcccaaggacaccctgatgatcagccggacccccgaa |
| | D265A/P329A | gtgacctgcgtggtggtggccgtgtcccacgaggaccctgaa |
| | (DAPA) | gtgaagttcaattggtacgtggacggcgtggaagtgcacaac |
| | Nucleic acid | gccaagaccaagcccagagaggaacagtacaacagcacctac |
| | sequence | cgggtggtgtccgtgctgaccgtgctgcaccaggactggctg |
| | | aacggcaaagagtacaagtgcaaagtctccaacaaggccctg |
| | | gccgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 15 | Human IgG1 Fc | apeaaggpsvflfppkpkdtlmisrtpevtcvvvdvkhedpe |
| | variant | vkfnwyvdgvevhnaktkpreeqynstyrvvsvltvlhqdwl |
| | L234A/L235A/ | ngkeykckvsnkalaapiektiskakgqprepqvytlppsrd |
| | S267K/P329A | eltknqvsltclvkgfypsdiavewesngqpennykttppvl |
| | (LALASKPA) | dsdgsfflyskltvdksrwqqgnvfscsvmhealhnhytqks |
| | Amino acid | lslspgk |
| | sequence | |
| 16 | Human IgG1 Fc | gcccctgaagccgccggcggaccctccgtgttcctgttcccc |
| | variant | ccaaagcccaaggacaccctgatgatcagccggacccccgaa |
| | L234A/L235A/ | gtgacctgcgtggtggtggacgtgaagcacgaggaccctgaa |
| | S267K/P329A | gtgaagttcaattggtacgtggacggcgtggaagtgcacaac |
| | (LALASKPA) | gccaagaccaagcccagagaggaacagtacaacagcacctac |
| | Nucleic acid | cgggtggtgtccgtgctgaccgtgctgcaccaggactggctg |
| | sequence | aacggcaaagagtacaagtgcaaagtctccaacaaggccctg |
| | | gccgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 17 | Human IgG1 Fc | apellggpsvflfppkpkdtlmisrtpevtcvvvavkhedpe |
| | variant | vkfnwyvdgvevhnaktkpreeqynstyrvvsvltvlhqdwl |
| | D265A/P329A/ | ngkeykckvsnkalaapiektiskakgqprepqvytlppsrd |
| | S267K (DAPASK) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 18 | Human IgG1 Fc | gcccctgaactgctgggcggaccctccgtgttcctgttcccc |
| | variant | ccaaagcccaaggacaccctgatgatcagccggacccccgaa |
| | D265A/P329A/ | gtgacctgcgtggtggtggccgtgaagcacgaggaccctgaa |
| | S267K (DAPASK) | gtgaagttcaattggtacgtggacggcgtggaagtgcacaac |
| | Nucleic acid | gccaagaccaagcccagagaggaacagtacaacagcacctac |
| | sequence | cgggtggtgtccgtgctgaccgtgctgcaccaggactggctg |
| | | aacggcaaagagtacaagtgcaaagtctccaacaaggccctg |
| | | gccgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 19 | Human IgG1 Fc | apellgapsvflfppkpkdtlmisrtpevtcvvvavshedpe |
| | variant | vkfnwyvdgvevhnaktkpreeqynstyrvvsvltvlhqdwl |
| | G237A/D265A/ | ngkeykckvsnkalaapiektiskakgqprepqvytlppsrd |
| | P329A (GADAPA) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 20 | Human IgG1 Fc | gcccctgaactgctgggcgccccctccgtgttcctgttcccc |
| | variant | ccaaagcccaaggacaccctgatgatcagccggacccccgaa |
| | G237A/D265A/ | gtgacctgcgtggtggtggccgtgtcccacgaggaccctgaa |
| | P329A (GADAPA) | gtgaagttcaattggtacgtggacggcgtggaagtgcacaac |
| | Nucleic acid | gccaagaccaagcccagagaggaacagtacaacagcacctac |
| | sequence | cgggtggtgtccgtgctgaccgtgctgcaccaggactggctg |
| | | aacggcaaagagtacaagtgcaaagtctccaacaaggccctg |
| | | gccgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 21 | Human IgG1 Fc | apellgapsvflfppkpkdtlmisrtpevtcvvvavkhedpe |
| | variant | vkfnwyvdgvevhnaktkpreeqynstyrvvsvltvlhqdwl |
| | G237A/D265A/ | ngkeykckvsnkalaapiektiskakgqprepqvytlppsrd |
| | P329A/S267K | eltknqvsltclvkgfypsdiavewesngqpennykttppvl |
| | (GADAPASK) | dsdgsfflyskltvdksrwqqgnvfscsvmhealhnhytqks |
| | Amino acid | lslspgk |
| | sequence | |
| 22 | Human IgG1 Fc | gcccctgaactgctgggcgccccctccgtgttcctgttcccc |
| | variant | ccaaagcccaaggacaccctgatgatcagccggacccccgaa |
| | G237A/D265A/P3 | gtgacctgcgtggtggtggccgtgaagcacgaggaccctgaa |
| | 29A/S267K | gtgaagttcaattggtacgtggacggcgtggaagtgcacaac |
| | (GADAPASK) | gccaagaccaagcccagagaggaacagtacaacagcacctac |
| | Nucleic acid | cgggtggtgtccgtgctgaccgtgctgcaccaggactggctg |
| | sequence | aacggcaaagagtacaagtgcaaagtctccaacaaggccctg |
| | | gccgcccccatcgagaaaaccatcagcaaggccaagggccag |
| | | ccccgcgagccccaggtgtacacactgccccccagccgggac |
| | | gagctgaccaagaaccaggtgtccctgacctgcctggtcaag |
| | | ggcttctaccccagcgatatcgccgtggaatgggagagcaac |
| | | ggccagcccgagaacaactacaagaccaccccccctgtgctg |
| | | gacagcgacggctcattcttcctgtacagcaagctgaccgtg |
| | | gacaagtcccggtggcagcagggcaacgtgttcagctgcagc |
| | | gtgatgcacgaggccctgcacaaccactacacccagaagtcc |
| | | ctgagcctgagccccggcaaa |
| 23 | Human IgG1 Fc | apellggpsvflfppkpkdtlmisrtpevtcvvvavshedpe |
| | variant | vkfnwyvdgvevhnaktkpreeqyastyrvvsvltvlhqdwl |
| | D265A/N297A/ | ngkeykckvsnkalaapiektiskakgqprepqvytlppsrd |
| | P329A (DANAPA) | eltknqvsltclvkgfypsdiavewesngqpennykttppvl |
| | Amino acid | dsdgsfflyskltvdksrwqqgnvfscsvmhealhnhytqks |
| | sequence | lslspgk |
| 24 | Human IgG1 Fc | gcccctgaactgctgggaggccctagcgtgttcctgttcccc |
| | variant | ccaaagcccaaggacaccctgatgatcagccggacccccgaa |
| | D265A/N297A/ | gtgacctgtgtggtggtggccgtgtctcacgaggaccctgaa |
| | P329A (DANAPA) | gtgaagtttaattggtacgtggacggcgtggaagtgcacaac |
| | Nucleic acid | gccaagaccaagcccagagaggaacagtacgccagcacctac |
| | sequence | cgggtggtgtccgtgctgacagtgctgcaccaggactggctg |
| | | aacggcaaagagtacaagtgcaaggtgtccaacaaggccctg |
| | | gccgctcccatcgagaaaaccatcagcaaggccaagggccag |
| | | ccccgcgaaccccaggtgtacacactgccccctagcagggac |
| | | gagctgaccaagaaccaggtgtccctgacctgcctcgtgaag |
| | | ggcttctacccctccgatatcgccgtggaatgggagagcaac |
| | | ggccagcccgagaacaactacaagaccaccccccctgtgctg |
| | | gactccgacggctcattcttcctgtacagcaagctgaccgtg |
| | | gacaagtcccggtggcagcagggcaacgtgttcagctgctcc |
| | | gtgatgcacgaggccctgcacaaccactacacccagaagtcc |
| | | ctgagcctgagccccggcaaa |

### Example 3: Biophysical Properties of Modified Antibodies

SPR-Binding of Modified Antibodies to Human Fc Gamma Receptors and Human C1q

Surface plasmon resonance (SPR) experiments were performed to analyze the interaction of human activating receptors FcγR1A, FcγR3A (V158) and human C1q with IgG1 WT and antibody-Fc variants. Binding kinetics and their relative binding affinities were explored. The binding affinity is an important characteristic of an interaction between an antibody and an antigen. The equilibrium dissociation constant (K_{D}) defines how strong the interaction is and therefore how much antibody-antigen complex is formed at equilibrium. The knowledge of the antibody characteristics is not only essential during selection of the best therapeutic antibody candidate, but also important to understand the in vivo behavior and potentially predict cellular immune responses. The aim is to generate antibody variants with little or no binding to Fc gamma receptors to reduce or eliminate effector function aiming to improve the safety of monoclonal antibody therapeutics. Binding to human C1q was evaluated. All SPR buffers were prepared using deionized water. The samples were prepared in running buffer PBS pH 7.4 with 0.005% Tween-20. SPR measurements were measured on a Biacore T200 (GE-Healthcare Life Sciences) controlled by Biacore T200 control software version 2.0.1. Surface plasmon resonance was conducted using a Biacore T200 to assess binding affinity of antibody IgG1 WT and variants to human Fc receptors, including FcγR1A, and FcγR3A (V158) and human C1q.

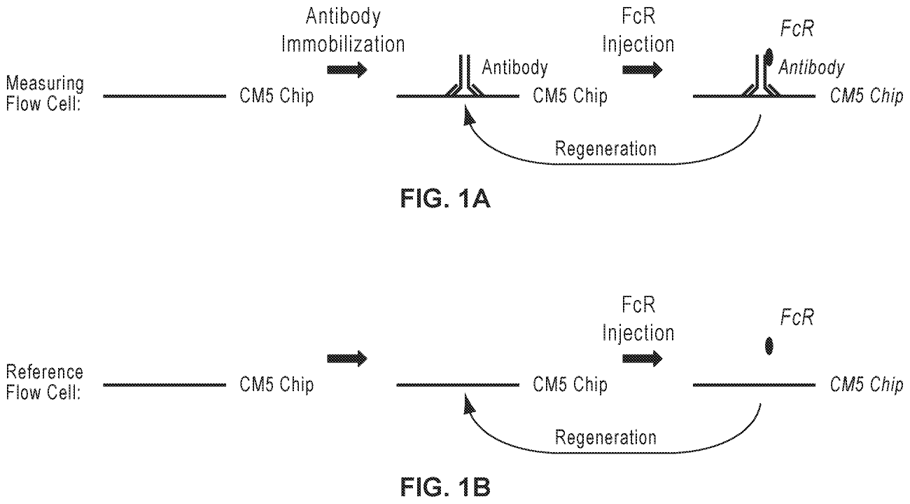

The antibodies were covalently immobilized on a CM5 sensor chip whereas Fc gamma receptors or human C1q served as analytes in solution (FIG. 1). For Fc gamma receptors binding evaluation (method 1), antibodies were diluted in 10 mM sodium acetate pH 4 and immobilized at a density of approximately 950 resonance units (RU's) on the CMS sensor chip applying a standard amine coupling procedure. Flow cell 1 was blank immobilized to serve as a reference. The kinetic binding data were collected by subsequent injections of 1:2 dilution series of the human Fc gamma receptors on all flow cells at a flow rate of 30 μl/min and at a temperature of 25° C. The Fc gamma receptors were diluted in the running buffer at concentrations ranging from 0.2 nM to 1000 nM (e.g. FcγR1A: 0.2 to 100 nM, FcγR3A V158: 1.95 to 1000 nM). The chip surface was regenerated using 20 mM glycine pH 2.0 solution after each measuring cycle. For human C1q binding evaluation (method 2), antibodies were diluted in 10 mM sodium acetate pH 4 and immobilized at a density of approximately 7000 resonance units (RU's) on the CMS sensor chip applying a standard amine coupling procedure. Flow cell 1 was blank immobilized to serve as a reference. The kinetic binding data were collected by subsequent injections of 1:2 dilution series of the human C1q on all flow cells at a flow rate of 30 μl/min and at a temperature of 25° C. The human C1q was diluted in the running buffer at concentrations ranging from 0.49 nM to 250 nM. The chip surface was regenerated using 50 mM NaOH solution after each measuring cycle. Zero concentration samples (blank runs) were measured for both methods to allow double-referencing during data evaluation.

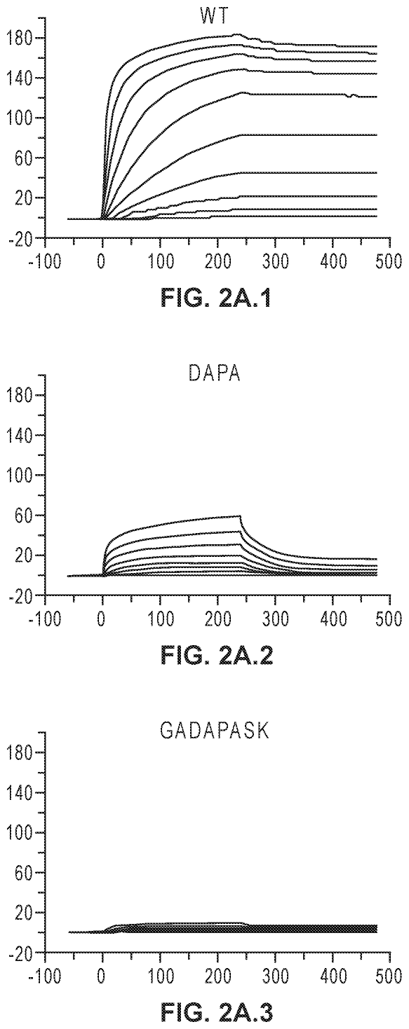

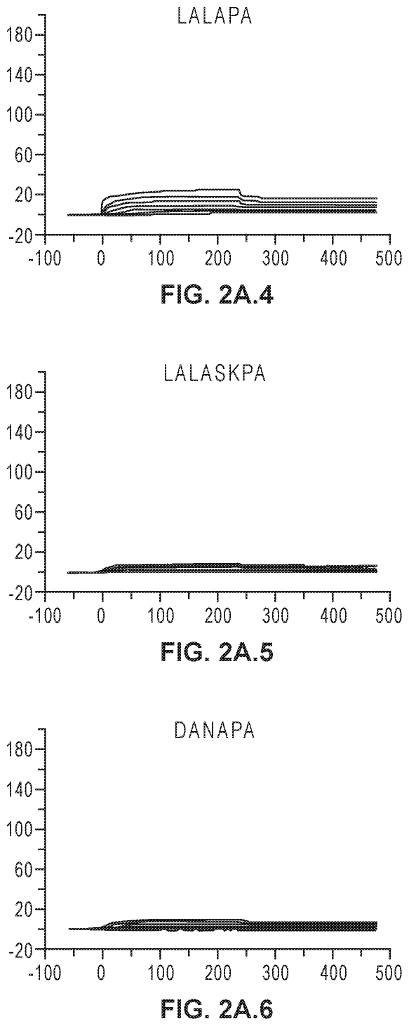

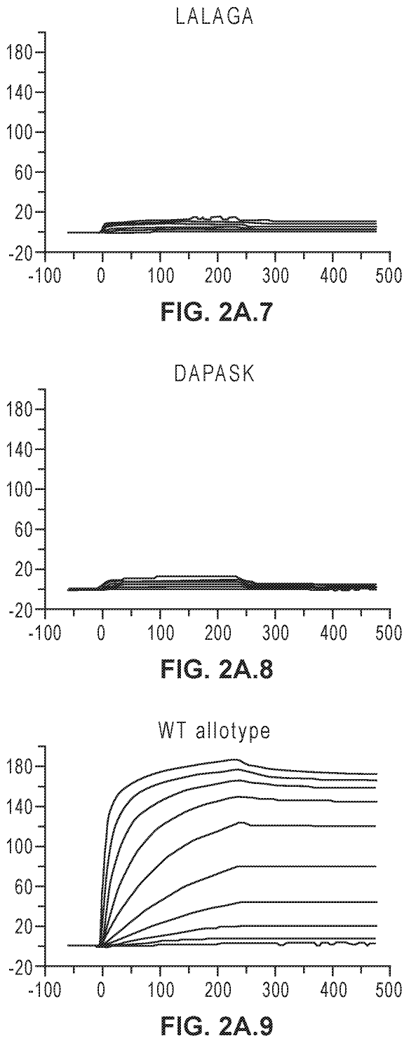

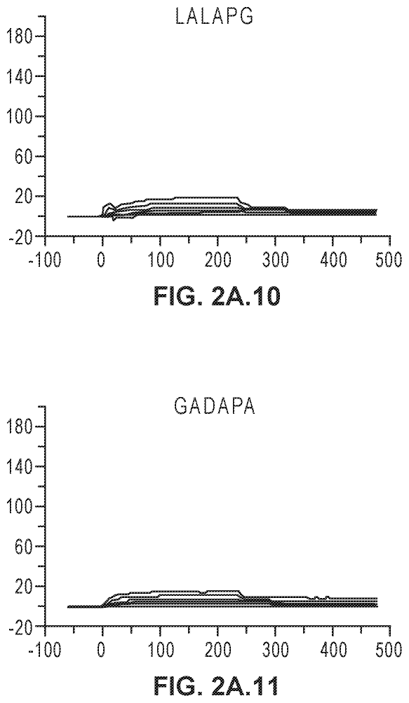

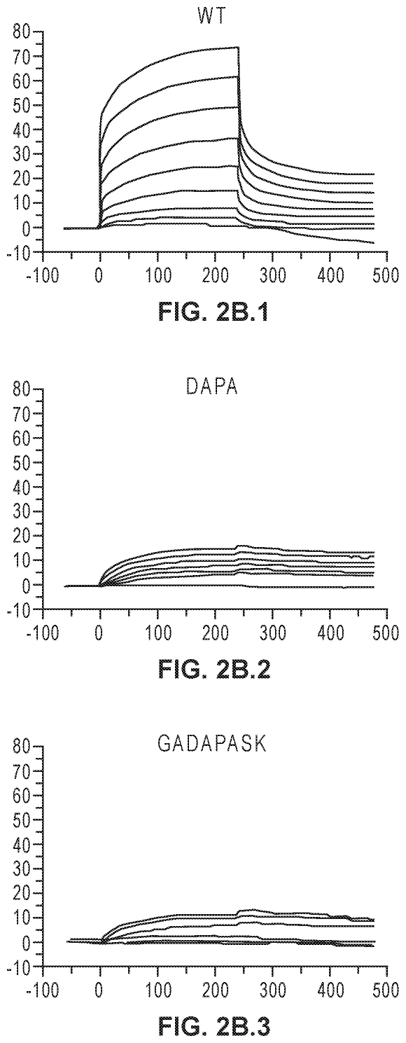

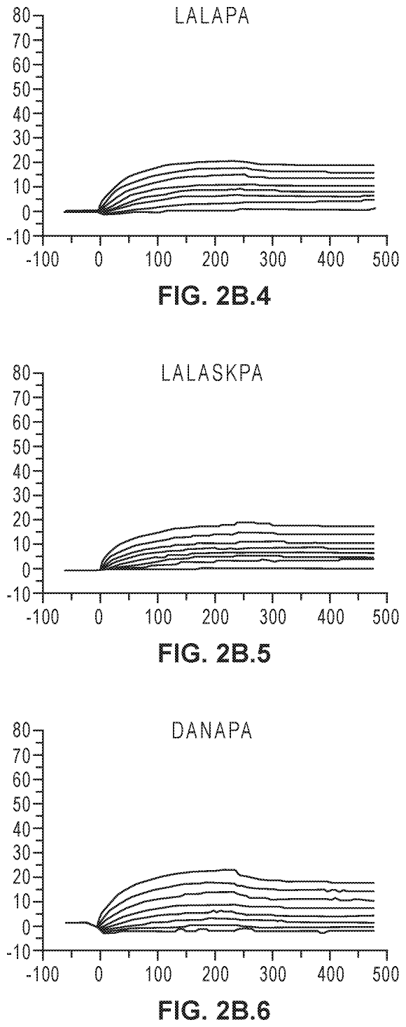

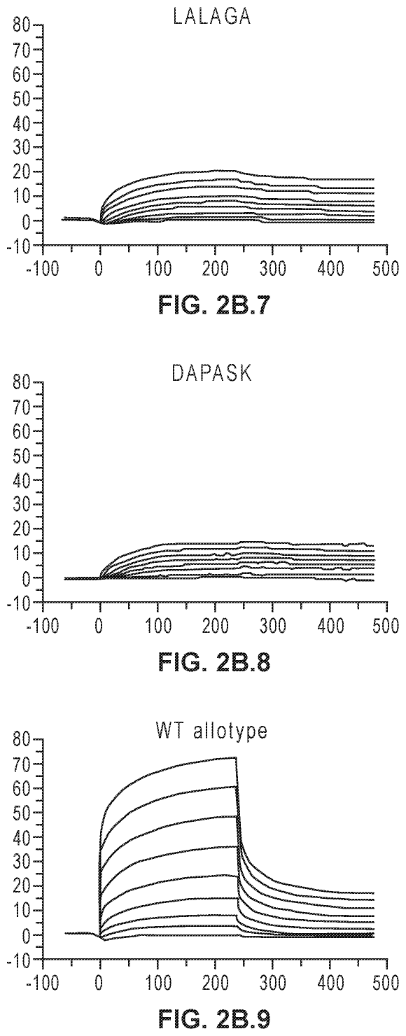

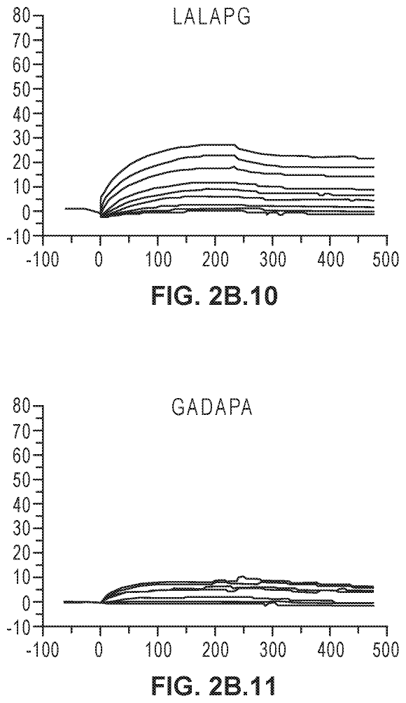

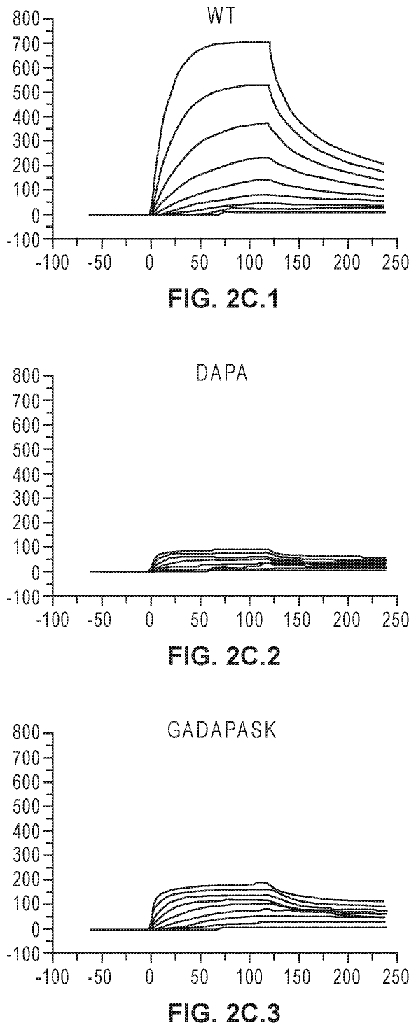

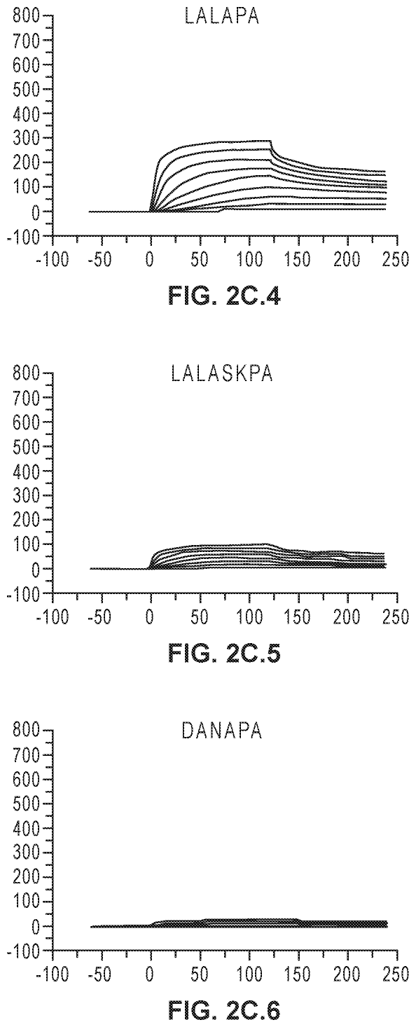

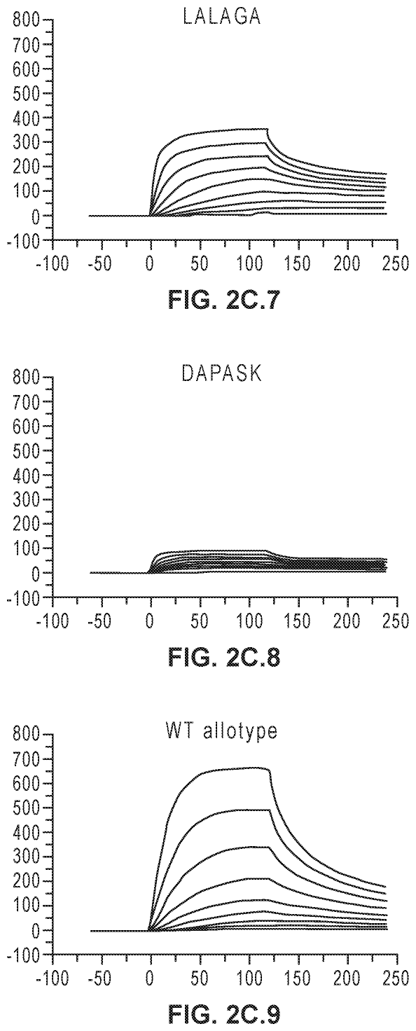

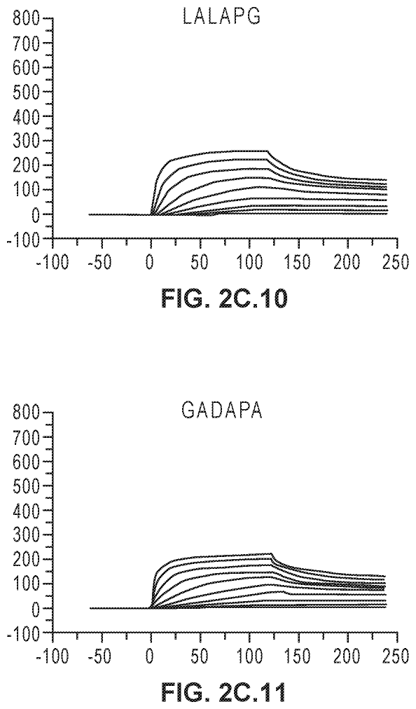

Data were evaluated using the Biacore T200 evaluation software. The raw data were double referenced, i.e. the response of the measuring flow cell was corrected for the response of the reference flow cell, and in a second step the response of a blank injection was subtracted. Then the sensorgrams were fitted by applying a 1:1 kinetic binding model to calculate dissociation equilibrium constants. In addition, the maximum response reached during the experiment was monitored. Maximum response describes the binding capacity of the surface in terms of the response at saturation. The maximum response values summarizing these interactions are given in Table 2. The SPR Biacore binding sensorgrams for each variant to each receptor were depicted in FIG. 2, Concentration range: 0.2nM-100nM for FcgR1, 7.8 nM-4000 nm for FcγR2A R131 d FcγR3A (V158 and F158). FIG. 2A shows representative sensorgrams and response plots of WT and variants towards FcgammaR1A (Concentration range: 0.2 nM-100 nM for human FcγR1A). FIG. 2B shows representative sensorgrams and response plots of WT and variants towards FcgammaR3A V158 (Concentration range: 1.95 nM-1000 nM for human FcγR3A V158). FIG. 2C shows representative sensorgrams and response plots of WT and variants towards human C1q (Concentration range: 0.49 nM-250 nM for human C1q). All IgG1 antibody-Fc variants inhibit the binding to Fc gamma receptors compare to WT (SEQ ID NO 1 and 3) and little or no residual binding was measured. All IgG1 antibody-Fc variants inhibit the binding to human C1q compare to WT (SEQ ID NO 1 and 3) as low residual binding was measured.

TABLE 2
Maximum responses of WT-Fc and variants towards human FcgammaRs
and C1q determined by surface plasmon resonance.

| | | Maximum | Maximum | Maximum |
|---|---|---|---|---|
| | | response at | response at | response at |
| | SEQ | 100 nM | 1000 nM | 250 nM |
| | ID | hFcgammaRIA | hFcgammaRIIIA | hC1q |
| Fc | NO | (RU) | V158 (RU) | (RU) |
| WT | 1 | 193 | 79 | 680 |
| WT allotype | 3 | 193 | 72 | 640 |
| DANAPA | 23 | 11 | 31 | 29 |
| DAPA | 13 | 63 | 15 | 85 |
| LALAPA | 7 | 26 | 19 | 270 |
| LALAPG | 11 | 18 | 33 | 245 |
| LALAGA | 9 | 15 | 25 | 335 |
| LALASKPA | 15 | 9 | 20 | 95 |
| DAPASK | 17 | 13 | 15 | 86 |
| GADAPA | 19 | 16 | 9 | 203 |
| GADAPASK | 21 | 10 | 12 | 168 |

### Example 4: Differential Scanning Calorimetry-Melting Temperature of Modified Antibodies

The thermal stability of engineered antibodies CH2 domains were compared using calorimetric measurements as shown in Table 3. calorimetric measurements were carried out on a differential scanning micro calorimeter (Nano DSC, TA instruments). The cell volume was 0.5 ml and the heating rate was 1° C./min. All proteins were used at a concentration of 1 mg/ml in PBS (pH 7.4). The molar heat capacity of each protein was estimated by comparison with duplicate samples containing identical buffer from which the protein had been omitted. The partial molar heat capacities and melting curves were analyzed using standard procedure. Thermograms were baseline corrected and concentration normalized. The silent version LALASKPA (70° C.) shows significantly better Tm compared to DANAPA (62° C.).

TABLE 3
Melting temperatures of WT-Fc and variants.

| Fc | Melting temperature (Tm) of CH2 domain |
|---|---|
| WT | 70 |
| WT allotype R214K | 70 |
| LALAPA | 70 |
| LALAGA | 70 |
| LALAPG | 70 |
| DAPA | 65 |
| LALASKPA | 70 |
| DAPASK | 65 |
| GADAPA | 65 |
| GADAPASK | 65 |
| DANAPA | 62 |

### Aggregation Propensity Post Capture of IgG1 anti-CD3 Sntibody and Fc Variants

Size exclusion chromatography measurements were performed to evaluate the aggregation propensity (% UMW) of IgG1 antibody and Fc modified derivatives. The produced and purified anti CD3 antibodies were applied to an analytical size exclusion chromatography column (SEC 200, GE Healthcare), equilibrated with PBS buffer pH 7.4. Results are summarized in Table 4.

TABLE 4
Higher molecular weight content (%) of anti-CD3 antibodies

| Fc | (%) higher molecular weight |
|---|---|
| WT | 7.3 |
| WT allotype R214K | 7.7 |
| LALAPA | 1.8 |
| LALAGA | 2.9 |
| LALAPG | 4.2 |
| DAPA | 3.9 |
| LALASKPA | 4.9 |
| DAPASK | 3.6 |
| GADAPA | 8.0 |
| GADAPASK | 6.8 |
| DANAPA | 5.9 |

### Example 5: Anti-CD3 NFAT Signalling Assay

Jurkat reporter gene assay (RGA) for the nuclear factor of activated T-cells (NFAT) pathway was performed using Jurkat NFAT luciferized (JNL) cells and THP-1 cells (ATCC, TIB202). THP-1 cells express FcγRI, FcγRII, and FcγRIII. Cells were co-incubated for 6 hours at 37° C., 5% CO₂ at a 5:1 effector to tumor ratio with each sample at the various concentrations depicted. An equal volume of ONE-Glo™ reagent (Promega, E6110) was added to the culture volume. Plate was shaken for 2 minutes, then incubated for an additional 8 minutes protected from light. For JNL+THP+IFNg experiment, THP-1 cells were pre-treated with 100 u/mL IFNg for 48 hours at 37° C., 5% CO₂ before co-culture. IFNg stimulation increases FcγRI expression. Luciferase activity was quantitated on the EnVision plate reader (PerkinElmer). Data was analyzed and fit to a 5 parameter-logistic curve using GraphPad Prism.

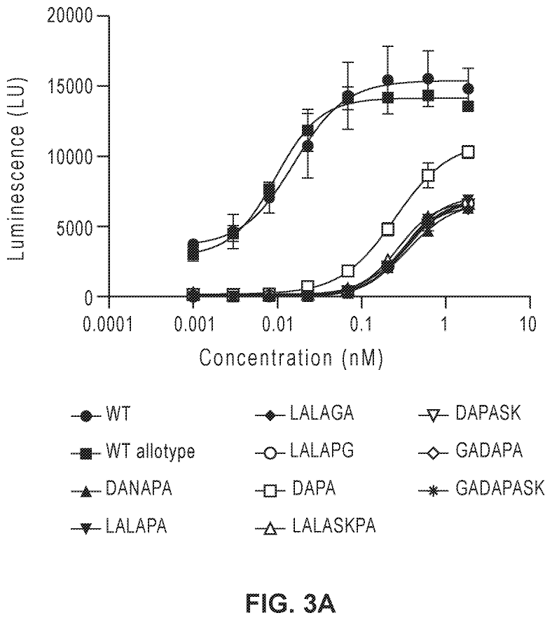

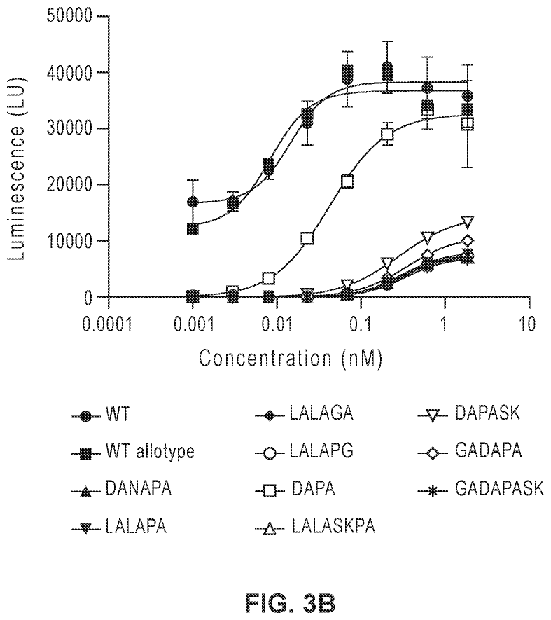

In both treatments, WT showed the greatest NFAT activity. All silencing mutation sets overall showed significantly dampened NFAT activation. In the RGA, performed without IFNg (FIG. 3A), all silencing mutation sets showed comparable T-cell activation with the exception of DAPA. When the THP-1 cells were preincubated with IFNg (FIG. 3B), the mutation sets showed lower activity, demonstrating strong Fc silencing, but some activity remaining in DAPA, LALAPA and GADAPA.

1. A binding molecule comprising a human IgG1 Fc variant of a wild-type human IgG1 Fc region and one or more antigen binding domains, wherein the Fc variant comprises a combination of amino acid substitutions and wherein the amino acid residues are numbered according to the EU index of Kabat.

2. The binding molecule of claim 1, wherein the Fc variant comprises the sequence of SEQ ID NO 15 or 21, or a sequence having at least 95%, 96%, 97%, 98%, or 99% homology thereto.

3. The binding molecule according to claim 1, wherein the binding molecule is a human or humanized IgG1 monoclonal antibody.

4. The binding molecule according to claim 1, wherein the binding molecule has reduced or undetectable binding affinity to a Fc gamma receptor compared to a polypeptide comprising the wild-type human IgG1 Fc region, optionally measured by surface plasmon resonance using a Biacore T200 instrument, wherein the Fc gamma receptor is selected from the group consisting of Fc gamma RIA and Fc gamma RIIIa V158 variant.

5. The binding molecule according to claim 1, wherein the antigen is a cell surface antigen.

6. The binding molecule according to claim 1, wherein the binding molecule has reduced or undetectable effector function compared to a polypeptide comprising the wild-type human IgG1 Fc region.

7. The binding molecule according claim 1, wherein the binding molecule is capable of binding to one or more antigens without triggering detectable antibody-dependent cell-mediated cytotoxicity (ADCC), antibody-dependent cellular phagocytosis (ADCP), or complement dependent cytotoxicity (CDC).

8. The binding molecule according to claim 1, wherein the binding molecule is a multi-specific antibody comprising binding domains for two or more antigens.

9. The binding molecule according to claim 8, wherein the binding molecule is a bi-specific antibody comprising binding domains for two antigens.

10. The binding molecule according to claim 8, wherein the Fc variant further comprises one or more knob-in-hole mutations.

11. The binding molecules of claim 1 for use in a method of treating a disease in an individual, wherein the effector function of the binding molecule is reduced or undetectable in the individual compared to the effector function induced by a polypeptide comprising the wild-type human IgG1 Fc region, the method comprising administering the binding molecule according to any one of claims 1-10 to the individual.

12. The binding molecules of claim 11, wherein the effector function is antibody-dependent cell-mediated cytotoxicity (ADCC).

13. The binding molecules of claim 11, wherein the effector function is antibody-dependent cellular phagocytosis (ADCP).

14. The binding molecules of claim 11, wherein the effector function is complement dependent cytotoxicity (CDC).

15. A composition comprising the binding molecule of claim 1.

16. The composition of claim 15, further comprising a pharmaceutically acceptable carrier.

17. An isolated polynucleotide comprising a sequence encoding the binding molecule of claim 1.

18. A vector comprising the polynucleotide of claim 17.

19. A host cell comprising the vector of claim 18.

20. A host cell comprising the polynucleotide of claim 17.


SEQUENCE LISTING (24 sequences)

SEQ ID NO: 1 - protein, 455 aa, Artificial Sequence
  source: /note=“Description of Artificial Sequence Synthetic polypeptide”
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 1365 bp, Artificial Sequence
  source: /note=“Description of Artificial Sequence Synthetic polynucleotide”
gaagtgcagctggtggaatctggcggcggactggtgcagcctggcggatctctgaagctgagctgtgccgccagcggcttcaccttcaacacctacgccatgaactgggtgcgccaggcctctggcaagggcctggaatgggtgggacggatcagaagcaagtacaacaattacgccacctactacgccgacagcgtgaaggaccggttcaccatcagccgggacgacagcaagagcaccctgtacctgcagatgaacagcctgaaaaccgaggacaccgccgtgtactactgcgtgcggcacggcaacttcggcaacagctatgtgtcttggtttgcctactggggccagggcaccctcgtgacagtgagctcagctagcaccaagggccccagcgtgttccccctggcgcccagcagcaagagcaccagcggcggcacagccgccctgggctgcctggtgaaggactacttccccgagccagtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagagagtggagcccaagagctgcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 3 - protein, 455 aa, Artificial Sequence
  source: /note=“Description of Artificial Sequence Synthetic polypeptide”
EVQLVESGGGLVQPGGSLKLSCAASGFTFNTYAMNWVRQASGKGLEWVGRIRSKYNNYATYYADSVKDRFTISRDDSKSTLYLQMNSLKTEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 1365 bp, Artificial Sequence
  source: /note=“Description of Artificial Sequence Synthetic polynucleotide”
gaagtgcagctggtggaatctggcggcggactggtgcagcctggcggatctctgaagctgagctgtgccgccagcggcttcaccttcaacacctacgccatgaactgggtgcgccaggcctctggcaagggcctggaatgggtgggacggatcagaagcaagtacaacaattacgccacctactacgccgacagcgtgaaggaccggttcaccatcagccgggacgacagcaagagcaccctgtacctgcagatgaacagcctgaaaaccgaggacaccgccgtgtactactgcgtgcggcacggcaacttcggcaacagctatgtgtcttggtttgcctactggggccagggcaccctcgtgacagtgagctcagctagcaccaagggccccagcgtgttccccctggcgcccagcagcaagagcaccagcggcggcacagccgccctgggctgcctggtgaaggactacttccccgagccagtgaccgtgtcctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgtcctgcccctgaactgctgggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 5 - protein, 215 aa, Artificial Sequence
  source: /note=“Description of Artificial Sequence Synthetic polypeptide”
QAVVTQEPSLTVSPGGTVTLTCRSSTGAVTTSNYANWVQQKPGQAPRGLIGGTNKRAPWTPARFSGSLLGDKAALTLSGAQPEDEAEYFCALWYSNLWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 6 - DNA, 645 bp, Artificial Sequence
  source: /note=“Description of Artificial Sequence Synthetic polynucleotide”
caggctgtcgtgacccaggaacctagcctgaccgtgtctcctggcggaaccgtgaccctgacctgtagatctagcacaggcgccgtgaccaccagcaactacgccaattgggtgcagcagaagcccggccaggctcctagaggactgatcggcggcaccaacaagagagccccttggacccctgccagattcagcggctctctgctgggagataaggccgccctgacactgtctggcgcccagcctgaggatgaggccgagtacttttgcgccctgtggtacagcaacctgtgggtgttcggcggaggcaccaagctgaccgtgctgggccagcctaaggccgctccctccgtgaccctgttcccccccagctccgaggaactgcaggccaacaaggccaccctggtgtgcctgatcagcgacttctaccctggcgccgtgaccgtggcctggaaggccgacagcagccccgtgaaggccggcgtggagacaaccacccccagcaagcagagcaacaacaagtacgccgccagcagctacctgagcctgacccccgagcagtggaagagccacagaagctacagctgccaggtcacccacgagggcagcaccgtggagaaaaccgtggcccccaccgagtgcagc

SEQ ID NO: 7 - protein, 217 aa, Artificial Sequence
  source: /note=“Description of Artificial Sequence Synthetic polypeptide”
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - DNA, 651 bp, Artificial Sequence
  source: /note=“Description of Artificial Sequence Synthetic polynucleotide”
gcccctgaagccgccggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctggccgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 9 - protein, 217 aa, Artificial Sequence
  source: /note=“Description of Artificial Sequence Synthetic polypeptide”
APEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - DNA, 651 bp, Artificial Sequence
  source: /note=“Description of Artificial Sequence Synthetic polynucleotide”
gcccctgaagccgccggcgccccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgcctgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 11 - protein, 217 aa, Artificial Sequence
  source: /note=“Description of Artificial Sequence Synthetic polypeptide”
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 651 bp, Artificial Sequence
  source: /note=“Description of Artificial Sequence Synthetic polynucleotide”
gcccctgaagccgccggcggaccctccgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctgggcgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 13 - protein, 217 aa, Artificial Sequence
  source: /note=“Description of Artificial Sequence Synthetic polypeptide”
APELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - DNA, 651 bp, Artificial Sequence
  source: /note=“Description of Artificial Sequence Synthetic polynucleotide”
gcccctgaactgctgggcggaccctccgtgttcctgttccccccaaagcccaaggacaccctgatgatcagccggacccccgaagtgacctgcgtggtggtggccgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctggccgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 15 - protein, 217 aa, Artificial Sequence
  source: /note=“Description of Artificial Sequence Synthetic polypeptide”
APEAAGGPSVFLFPPKPKDTLMISRTPEVTCVVVDVKHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - DNA, 651 bp, Artificial Sequence
  source: /note=“Description of Artificial Sequence Synthetic polynucleotide”
gcccctgaagccgccggcggaccctccgtgttcctgttccccccaaagcccaaggacaccctgatgatcagccggacccccgaagtgacctgcgtggtggtggacgtgaagcacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctggccgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 17 - protein, 217 aa, Artificial Sequence
  source: /note=“Description of Artificial Sequence Synthetic polypeptide”
APELLGGPSVFLFPPKPKDTLMISRTPEVTCVVVAVKHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 651 bp, Artificial Sequence
  source: /note=“Description of Artificial Sequence Synthetic polynucleotide”
gcccctgaactgctgggcggaccctccgtgttcctgttccccccaaagcccaaggacaccctgatgatcagccggacccccgaagtgacctgcgtggtggtggccgtgaagcacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctggccgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 19 - protein, 217 aa, Artificial Sequence
  source: /note=“Description of Artificial Sequence Synthetic polypeptide”
APELLGAPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - DNA, 651 bp, Artificial Sequence
  source: /note=“Description of Artificial Sequence Synthetic polynucleotide”
gcccctgaactgctgggcgccccctccgtgttcctgttccccccaaagcccaaggacaccctgatgatcagccggacccccgaagtgacctgcgtggtggtggccgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctggccgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 21 - protein, 217 aa, Artificial Sequence
  source: /note=“Description of Artificial Sequence Synthetic polypeptide”
APELLGAPSVFLFPPKPKDTLMISRTPEVTCVVVAVKHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 651 bp, Artificial Sequence
  source: /note=“Description of Artificial Sequence Synthetic polynucleotide”
gcccctgaactgctgggcgccccctccgtgttcctgttccccccaaagcccaaggacaccctgatgatcagccggacccccgaagtgacctgcgtggtggtggccgtgaagcacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaagtctccaacaaggccctggccgcccccatcgagaaaaccatcagcaaggccaagggccagccccgcgagccccaggtgtacacactgccccccagccgggacgagctgaccaagaaccaggtgtccctgacctgcctggtcaagggcttctaccccagcgatatcgccgtggaatgggagagcaacggccagcccgagaacaactacaagaccaccccccctgtgctggacagcgacggctcattcttcctgtacagcaagctgaccgtggacaagtcccggtggcagcagggcaacgtgttcagctgcagcgtgatgcacgaggccctgcacaaccactacacccagaagtccctgagcctgagccccggcaaa

SEQ ID NO: 23 - protein, 217 aa, Artificial Sequence
  source: /note=“Description of Artificial Sequence Synthetic polypeptide”
APELLGGPSVFLFPPKPKDTLMISRTPEVTCVVVAVSHEDPEVKFNWYVDGVEVHNAKTKPREEQYASTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 651 bp, Artificial Sequence
  source: /note=“Description of Artificial Sequence Synthetic polynucleotide”
gcccctgaactgctgggaggccctagcgtgttcctgttccccccaaagcccaaggacaccctgatgatcagccggacccccgaagtgacctgtgtggtggtggccgtgtctcacgaggaccctgaagtgaagtttaattggtacgtggacggcgtggaagtgcacaacgccaagaccaagcccagagaggaacagtacgccagcacctaccgggtggtgtccgtgctgacagtgctgcaccaggactggctgaacggcaaagagtacaagtgcaaggtgtccaacaaggccctggccgctcccatcgagaaaaccatcagcaaggccaagggccagccccgcgaaccccaggtgtacacactgccccctagcagggacgagctgaccaagaaccaggtgtccctgacctgcctcgtgaagggcttctacccctccgatatcgccgtggaatgggagagcaacggccagcccgagaacaactacaagaccaccccccctgtgctggactccgacggctcattcttcctgtacagcaagctgaccgtggacaagtcccggtggcagcagggcaacgtgttcagctgctccgtgatgcacgaggccctgcacaaccactacacccagaagtccctgagcctgagccccggcaaa